(19) United States
(12) Patent Application Publication
(10) Pub. No.: US 2005/0256313 A1
(43) Pub. Date: Nov. 17, 2005

(54) Co-surfactants based on aldehydes

(75) Inventors: Ralf Norenberg, Ingelheim (DE); Monica Fernandez Gonzalez, Heidelberg (DE); Norbert Wagner, Mutterstadt (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/521,360

(22) PCT Filed: Jul. 17, 2003
(86) PCT No.: PCT/EP03/07766

§ 371 Date: Jan. 14, 2005

(30) Foreign Application Priority Data
Jul. 18, 2002 (DE) 102 32 780.7

(51) Int. Cl. C07D 491/04; C07D 317/08

(52) U.S. Cl. 548/217; 558/445; 549/229

(57) ABSTRACT

The present invention relates to cyclic cosurfactants which are produced by condensation reaction of C₃-C₆-aldehydes with polyfunctional alcohols, amines, thiols or carboxylic acids and have the formulae I or II as given in the description. The cosurfactants are suitable for use in household detergents, household cleaners, body-cleansing compositions and bodycare compositions.

The present invention relates to condensation products of aldehydes with alcohols which have at least one other function from the group of hydroxyl, diol, amino and carboxyl functions.

Surfactants are so-called amphiphilic molecules which have a hydrophobic moiety and a hydrophilic moiety in their molecular structure. As a result of this property, surfactants are able to form interfacial films and so-called micelles. These are aggregates of surfactants which form in aqueous solutions and can assume various forms (spheres, rods, disks). Micelles form above a certain concentration, the so-called critical micelle formation concentration (CMC). In addition, amphiphilic molecules have the property of forming interfacial films between hydrophobic and hydrophilic phases and thus, for example, having an emulsifying or foaming action.

Cosurfactants likewise have amphiphilic properties, although these are insufficient for being able to form micelles and interfacial films on their own. However, they are intercalated between the surfactants and bring about an increase in the packing density of the amphiphiles (surfactants and cosurfactants) in the structures formed thereby, such as micelles or interfaces. As a result, not only are the critical micelle formation concentration and the surface tension reduced, but also the interfacial tension between the aqueous surfactant solution and nonpolar substances such as, for example, oils, meaning that the absorption capacity of the surfactant system for these substances increases to the point of the formation of microemulsions. This results in a high solubilizing and emulsifying power, a higher cleaning capacity, and an increased stability of the emulsions and foams. If cosurfactants are used, micelles can be formed at a significantly lower surfactant concentration.

Further effects which are brought about as a result of the use of the cosurfactants and the resulting enhanced aggregation tendency of the amphiphiles are known. This is, firstly, the aggregation transformation of spherical to anisometric micellar associates. This structural change in the micelles has effects on the rheology of the solutions containing the micelles, in particular in dilute solutions. At the same time, in the phase diagram, there is a shift of liquid crystalline structures present to lower concentrations, as a result of which a preferred formation of gel phases with higher packing density is observed. Consequently, even at concentrations of significantly <10% by weight, lamellar micelle structures arise which are otherwise observed only at significantly higher concentrations. A further interesting phenomenon is the formation, in addition to the known liquid crystalline gel phases, of novel superstructures which have interesting application properties. Of particular interest here are vesicular phases and also so-called L₃ phases which have a sponge-like construction and have microemulsion-like properties. They can be used in dilute concentration ranges to adjust the viscosity.

The prior art describes a number of compounds or classes of compounds which are suitable as cosurfactants.

C₅-C₁₀-alcohols exhibit advantageous properties, but are often not used due to their characteristic odor.

Alcohols with low degrees of ethoxylation, such as, for example, lauryl alcohol ethoxylates with low degrees of ethoxylation, diethylene glycol monohexyl ether or propylene glycol butyl ether, can lead to improved emulsifying power or foam stability in some surfactant systems, but have too low a polarity of the head group for surfactant formulations with a high anionic surfactant content.

Fatty acid ethanolamines are used, for example, for adjusting the viscosity in shampoos. However, they are suspected of forming nitrosamines.

G. J. Smith describes in Seifen, Ölen, Fette, Wachse, 105 (1979, pages 319 ff and 345 ff) the use of alkylamine oxides as cosurfactant in various applications. These too are suspected of containing nitrosamines. Through a lengthy, complex preparation technology, that can be largely avoided.

Analogously to the amine oxides, other zwitterionic surfactants, such as, for example, sulfobetaines or carboxylammoniobetaines, can also be used as cosurfactant. With these products, the formation of gel phases has proven to be very poor. Instead, however, they have the application advantage that the skin irritancy of corresponding surfactant mixtures is reduced.

WO 98/00418 discloses alkylene carbonates and their use as cosurfactants.

In the applications known to date, the ratio of cosurfactants to surfactants used varies, depending on the application, from about 1:20 to 1:2. In some cases, such as, for example, alkylamine oxides, the cosurfactant may also be used in higher concentrations.

It is an object of the present invention to provide compounds suitable as cosurfactants which do not have said disadvantages, in particular exhibit better cost efficiency, and are environmentally compatible and free from risks for humans.

We have found that this object is achieved by cyclic aldehyde derivatives of the formulae 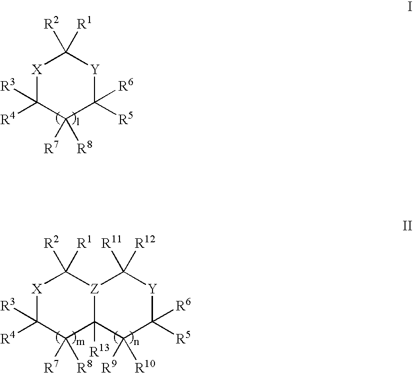
in which the symbols X, Y, Z and R¹ to R¹³ have the following meanings:
R¹ is hydrogen or a linear or branched, substituted or unsubstituted C₃-C₂₉-alkyl group or a linear or branched, substituted or unsubstituted C₃-C₂₉-alkenyl group, where one or more carbon atoms in the alkyl or alkenyl chain may be replaced by —O—, —NR¹⁴, —C(O)NR¹⁵— or —S— and —O—O— and —S—S— are excluded;
R² is hydrogen or —CH₃;
R³, R⁴, R⁵ and R⁶ are, independently of one another, chosen from the group of substituents consisting of: H; —CN; —C(O)OH; —C(O)OR¹⁸; —C(O)NR¹⁹R²⁰; C₆H₅, in which one or more hydrogens may be replaced by substituents; 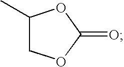
and C₁-C₅-alkyl groups which, at any desired point in the chain, may have 1 to 4 substituents from the group —OH; —SH; —CN; NR¹⁶R¹⁷; —OR²²; or 1 to 2 substituents from the group consisting of —C(O)OH; —C(O)OR¹⁸; —C(O)NR¹⁹R²⁰; —OSO₃⁻; —SO₃⁻; —OPO₃²⁻; OPO(OR²¹)₂; C₆H₅, in which one or more hydrogens may be replaced by substituents; and 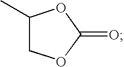
   or
one of the substituent pairs R³, R⁴ and R⁵, R⁶ is ═O;
R⁷, R⁸, R⁹ and R¹⁰ are, independently of one another, chosen from the group of substituents consisting of: H; —CN; —NR¹⁶R¹⁷; —C(O)OH; —C(O)OR¹⁸; —C(O)NR¹⁹R²⁰; —OSO₃⁻; —OPO₃²⁻; OPO(OR²¹)₂; C₆H₅, in which one or more hydrogens may be replaced by substituents; 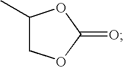
and C₁-C₅-alkyl groups which, at any desired point on the chain, may have 1 to 4 substituents from the group —OH; —SH; —CN; NR¹⁶R¹⁷; —OR²²; or 1 to 2 substituents from the group consisting of —C(O)OH; —C(O)OR¹⁸; —C(O)NR¹⁹R²⁰; —OSO₃⁻; —SO₃⁻; —OPO₃²⁻; OPO(OR²¹)₂; C₆H₅, in which one or more hydrogens may be replaced by substituents; and 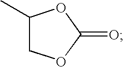
R¹¹ has, independently, the same meaning as R¹;
R¹² has, independently, the same meaning as R²;
R¹³ has, independently, the same meaning as R³, R⁴, R⁵ or R⁶;
R¹⁴ is a linear or branched C₁-C₄-alkyl group;
R¹⁵ is hydrogen or a linear or branched C₁-C₄-alkyl group;
R¹⁶, R¹⁷ are, independently of one another, hydrogen or a linear or branched C₁-C₄-alkyl group;
R¹⁸ is chosen from the group consisting of C₁-C₆-alkyl groups and ethyleneoxy groups —(CH₂—CH₂O—)ₚ;
R¹⁹, R²⁰ have, independently, the same meaning as R¹⁶, R¹⁷;
R²¹ is a C₁-C₄-alkyl group or —C₆H₅;
R²² is chosen from the group consisting of C₁-C₁₀-alkyl groups, acyl groups —C(O)R²³ and the group consisting of ethyleneoxy groups —(CH₂—CH₂O—)_{q}, propyleneoxy groups —(CH(CH₃)—CH₂O—)ᵣ, butyleneoxy groups —(C₄H₉O—)ₛ, and alkyleneoxy groups containing at least two of the abovementioned groups in the form of block or random copolymers and containing a total of at most 15 alkyleneoxy units;
R²³ is a C₁-C₁₈-alkyl group;
X and Y in formula I and II are, independently of one another, O, S, or NR²⁴, Z in formula II is N;
R²⁴ is hydrogen or a C₁-C₄-alkyl group;
l, m and n are, independently of one another, 0 or 1;
p is an integer from 1 to 15;
q is an integer from 1 to 15;
r is an integer from 1 to 15;
s is an integer from 1 to 15;
and where the aliphatic moiety of the compounds of the formula I or II which does not originate from the feed aldehyde, in cases where X, Y are ═O and R³, R⁴ or R⁵, R⁶ are ═O, must have at least 2 carbon atoms, and in all other cases must have at least 3 carbon atoms.

The compounds of the formulae I and II are highly suitable for use as cosurfactants in the customary washing and cleaning formulations known to the person skilled in the art.

The compounds according to the formulae I and II are prepared by condensation of aldehydes with polyfunctional compounds whose functional groups are chosen from the group of hydroxyl, thiol, carboxyl and primary and secondary amino functions. There must be at least two functional groups, which may be identical or different, present in the polyfunctional compound.

The aldehydes to be used are linear or branched aliphatic C₄-C₃₀-aldehydes, preferably C₆-C₁₈-aldehydes. These aldehydes have an average degree of branching of from 0 to 2.5, preferably 0.2 to 1.6. The degree of branching here is defined as (number of methyl groups per molecule) −1. Since the aliphatic chain radical of the aldehyde joined to the carbonyl function corresponds to the radical R¹ in the formulae I and II, this last-named radical also has a corresponding degree of branching. The alkyl chain can have further substituents which increase the suitability of the molecule as a cosurfactant, or at least do not adversely affect it. Such substituents are known to the person skilled in the art. Preferably, no further substituents are present on the alkyl chain. Examples of aldehydes which can be used include butanal, pentanal, hexanal, heptanal, octanal, nonanal, decanal, undecanal, dodecanal, tridecanal, tetradecanal and hexadecanal. For all of the abovementioned aldehydes, it is possible to use either the unbranched n-form or branched isomers. In general, use is made of isomer mixtures of the aldehydes used which have the desired average degree of branching.

It is also possible to use mixtures of aldehydes of varying carbon number and to use the resulting product mixtures as cosurfactants. This embodiment is preferred in accordance with the invention. Particular preference is given here to the use of a mixture of C₁₂-C₁₄-aldehydes.

Also preferred in accordance with the invention is the use of the so-called Guerbet aldehydes and their unsaturated analogs. These are aldehydes with branching in the 2 position. Examples include 2-ethylhexanal, 2-ethylhex-2-enal, 2-propylhexanal, 2-propylheptanal, 2-propylhept-2-enal, 2-butyloctanal, 2-butyloct-2-enal, 2-pentylnonanal and 2-pentylnon-2-enal. Saturated aldehydes are preferred.

The polyfunctional starting materials to be reacted with the aldehyde are the substances listed below.

Polyols:

Suitable polyols are linear and branched aliphatic C₃-C₆-polyols with at least two hydroxyl functions, preferably 2 to 5 hydroxyl functions, in particular 2 to 4 hydroxyl functions. As well as the hydroxyl functions, further functional groups may be present which are chosen from the group —SH; —CN; NR¹⁶R¹⁷; —C(O)OH; —C(O)OR¹⁸; —C(O)NR¹⁹R²⁰; —OSO₃⁻; —SO₃⁻; —OPO₃²⁻; OPO(OR²¹)₂; —OR²²; C₆H₅, in which one or more hydrogens may be replaced by substituents; and

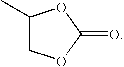

Examples of suitable polyols include glycerol, tartaric acid, diethyl tartrate, trimethylolpropane, fructose, cyclohexanediol, sucrose, tetrahydroxybutane.

Alkanolamines:

Suitable alkanolamines are linear and branched aliphatic C₃-C₆-alkanolamines with at least one primary or secondary amino function and one hydroxyl function. A maximum of 4 further hydroxyl or amino functions may be present, preferably the alkanolamine has exactly one amino function. Further substituents may be present from the group: —SH; —CN; —C(O)OH; —C(O)OR¹⁸; —C(O)NR¹⁹R²⁰; —OSO₃⁻; —SO₃⁻; —OPO₃²⁻; OPO(OR²¹)₂; —OR²²; —C₆H₅, in which one or more hydrogens may be replaced by substituents; and

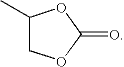

Examples of suitable alkanolamines include trimethylolmethylamine, diethanolamine, propanolamine, dipropanolamine, amino sugars, amino acids, such as, for example, sarcosine, serine and threonine.

Thiols:

Suitable thiols are linear and branched aliphatic C₃-C₆-thiols with one thiol function and one hydroxyl function. Four further hydroxyl or thiol functions may be present. Preferably, the thiol has exactly one SH function. Further substituents may be present from the group: —OH; —SH; —CN; NR¹⁶R¹⁷; —C(O)OH; —C(O)OR¹⁸; —C(O)NR¹⁹R²⁰; —OSO₃⁻; —SO₃⁻; —OPO₃²⁻; OPO(OR²¹)₂; —OR²²; —C₆H₅, in which one or more hydrogens may be replaced by substituents; and

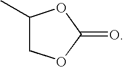

Examples of suitable thiols are mercaptoethanol, mercaptolactic acid, mercaptoglycolic acid, thiosalicylic acid, mercaptosuccinic acid, 3-mercapto-1,2-propanediol, cysteine, N-acetylcysteine, 3-mercaptopropionic acid, penicillamine, dithiothreitol.

Hydroxycarboxylic Acids:

Suitable hydroxycarboxylic acids are linear and branched aliphatic C₃-C₆-hydroxycarboxylic acids with one hydroxyl function and one carboxyl function. Four further hydroxyl or carboxyl functions may be present. Preferably, the molecule has exactly one carboxyl function. Further substituents may be present from the group: —SH; —CN; NR¹⁶R¹⁷; —C(O)OR¹⁸; —C(O)NR¹⁹R²⁰; —OSO₃⁻; —SO₃⁻; —OPO₃²⁻; OPO(OR²¹)₂; —OR²²; —C₆H₅, in which one or more hydrogens may be replaced by substituents; and

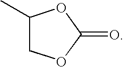

Examples of suitable hydroxycarboxylic acids include lactic acid, citric acid, glycolic acid, tartaric acid, glyceric acid, malic acid and salicylic acid.

Diamines:

It is possible to use linear and branched aliphatic C₃-C₆-diamines having 2 to 6 primary or secondary amino functions. Preferably, the diamines have 2 to 4 primary or secondary amino functions. Further substituents may be present from the group: —OH; —SH; —CN; —C(O)OH; —C(O)OR¹⁸; —C(O)NR¹⁹R²⁰; —OSO₃⁻; —SO₃⁻; —OPO₃²⁻; OPO(OR²¹)₂; —OR²²; —C₆H₅, in which one or more hydrogens may be replaced by substituents; and

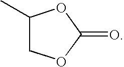

Examples of suitable diamines include propylenediamine, triethylenetriamine, triethylenetetramine, N-aminopropylethylenediamine (N₃-amine) and N,N′-bis(aminopropyl)ethylenediamine (N₄ amine), hydroxyethylethylenediamine.

Aminothiols:

Suitable aminothiols are linear and branched aliphatic C₃-C₆-aminothiols with at least one primary or secondary amino function and one thiol function. Preferably, the amino thiol used has exactly one primary or secondary amino function and exactly one thiol function. Further substituents may be present from the group: —OH; —CN; —C(O)OH; —C(O)OR¹⁸; —C(O)NR¹⁹R²⁰; —OSO₃⁻; —SO₃⁻; —OPO₃²⁻; OPO(OR²¹)₂; —OR²²; —C₆H₅, in which one or more hydrogens may be replaced by substituents; and

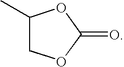

Examples include cysteine and tyrosine and mercaptopropylamine.

Amino Acids:

Suitable amino acids are linear and branched aliphatic C₃-C₆-amino acids with at least one primary or secondary amino function and one carboxyl function. Preferably, the amino acids used according to the invention contain one primary or secondary amino function. Examples include iminodiacetic acid HN(CH₂CO₂H)₂ and ethylenediamine triacetic acid, and the amino acids alanine, arginine, asparagine, aspartic acid, cysteine, glutamine, glutaminic acid, glycine, histidine, isoleucine, leucine, lysine, methionine, phenylalanine, proline, serine, threonine, tryptophan, tyrosine, valine and N-phosphonomethylglycine.

Dithiols:

Suitable dithiols are linear and branched aliphatic C₃-C₆-dithiols with at least two thiol functions. Preferably, the dithiol has exactly two thiol functions. Further substituents may be present from the group: —OH; —CN; NR¹⁶R¹⁷; —C(O)OH; —C(O)OR¹⁸; —C(O)NR¹⁹R²⁰; —OSO₃⁻; —SO₃⁻; OPO₃²⁻; OPO(OR²¹)₂; —OR²²; —C₆H₅, in which one or more hydrogens may be replaced by substituents; and

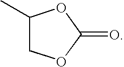

The alkyleneoxy substituents which may be present in the cosurfactants of the formulae I and II used according to the invention (see, for example, definition of R²²) may be pure ethyleneoxy, propyleneoxy or butyleneoxy substituents. Butyleneoxy substituents may be those derived from 1-butene, 2-butene or isobutene. Within the group of butyleneoxy substituents, preference is given to the group —(CH₂CH(C₂H₅)—O) derived from 1-butene. These substituents can, however, also consist of mixtures of ethyleneoxy, propyleneoxy or butyleneoxy units, for example as a random oligo/polymer or as a block copolymer. Preferably, said alkyleneoxy substituents are pure ethyleneoxy groups or terminally capped ethyleneoxy groups.

If an individual compound is present which contains alkyleneoxy substituents, then this contains a whole-number amount of alkyleneoxy units in the amounts given in the description. If mixtures of different compounds with alkyleneoxy substituents are present, these are often mixtures in which the amount of alkyleneoxy substituents is, on average, no longer a whole number.

—C₆H₅ designates a phenyl group.

Preferred molecules of the formulae I and II are those in which one or more of the symbols X, Y and Z, one or more of the substituents R¹ to R¹³, and one or more of the symbols l, m and n have the following meanings:
R¹ is a linear or branched C₅-C₁₇-alkyl group or a linear or branched C₃-C₁₇-alkenyl group, where one or more carbon atoms in the alkyl chain may be replaced by O or NR¹⁴ and —O—O— is excluded;
R² is —H;
R³, R⁴, R⁵ and R⁶ are, independently of one another, chosen from the group consisting of: —H; —C(O)OH; —C(O)OR¹⁸; 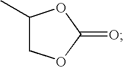
and C₁-C₅-alkyl groups which, at any desired position on the chain, may have 1 or 2 substituents from the group —OH; —CN; NR¹⁶R¹⁷; —OR²²; or 1 substituent from the group consisting of —C(O)OH; —C₆H₅, in which one or more hydrogens may be replaced by substituents; and 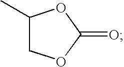
   or
one of the substituent pairs R³, R⁴ and R⁵, R⁶ is ═O;
R⁷, R⁸, R⁹ and R¹⁰ are, independently of one another, chosen from the group consisting of: —H; —NR¹⁶R¹⁷; —C(O)OH; —C(O)OR¹⁸; 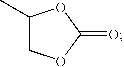
and C₁-C₅-alkyl groups which, at any desired position on the chain, may have 1 or 2 substituents from the group —OH; —CN; NR¹⁶R¹⁷; —OR²²; or 1 substituent from the group consisting of —C(O)OH; —C₆H₅, in which one or more hydrogens may be replaced by substituents; and 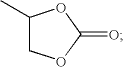
R¹¹ has, independently, the same meaning as R¹;
R¹² has, independently, the same meaning as R²;
R¹³ has, independently, the same meaning as R³, R⁴, R⁵ or R⁶;
R¹⁴ is a linear or branched C₁-C₄-alkyl group;
R¹⁶, R¹⁷ are, independently of one another, hydrogen or a linear or branched C₁-C₄-alkyl group;
R¹⁸ is chosen from the group consisting of C₁-C₆-alkyl groups and ethyleneoxy groups —(CH₂—CH₂O—)ₚ;
R²² is chosen from the group consisting of C₁-C₄-alkyl groups, acyl groups —C(O)R²³ and the group consisting of ethyleneoxy groups —(CH₂—CH₂O—)_{q}, propyleneoxy groups —(CH(CH₃)—CH₂O—)ᵣ and butyleneoxy groups —(C₄H₉O—)ₛ, and mixed alkyleneoxy groups;
R²³ is a C₁-C₁₈-alkyl group;
X and Y in the formula I and II are, independently of one another, O or NR²⁴, Z in formula II is N;
R²⁴ is hydrogen or a C₁-C₄-alkyl group;
l, m and n are, independently of one another, 0 or 1;
p is an integer from 1 to 15;
q is an integer from 1 to 10;
r is an integer from 1 to 10;
s is an integer from 1 to 10;
and where the aliphatic moiety of the compounds of the formula I or II which does not originate from the feed aldehyde, in cases where X, Y are ═O and R³, R⁴ or R⁵, R⁶ are ═O, must have at least 2 carbon atoms, and in all other cases must have at least 3 carbon atoms.

In this connection, it is preferred if all of the substituents R¹ to R¹³ and symbols X, Y and Z and l, m, and n have the meanings given above.

More preferred are also the compounds of the formulae I and II, in which one or more of the symbols X, Y, Z and l, m, and n and also one or more of the substituents R¹ to R¹³ have the following meanings:
R¹ is a linear or branched C₅-C₁₇-alkyl group or a linear or branched C₅-C₁₇-alkenyl group;
R² is —H;
R³, R⁴, R⁵ and R⁶ are, independently of one another, chosen from the group consisting of: —H; —C(O)OH; and C₁-C₃-alkyl groups which can have, at any desired point on the chain, 1 or 2 substituents from the group —OH; —NR¹⁶R¹⁷; —OR²²; or 1 substituent from the group consisting of —C(O)OH; and 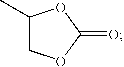
   or
one of the substituent pairs R³, R⁴ and R⁵, R⁶ is ═O;
R⁷, R⁸, R⁹ and R¹⁰ are, independently of one another, chosen from the group consisting of: —H; —NR¹⁶R¹⁷; —C(O)OH; and C₁-C₃-alkyl groups which, at any desired point on the chain, can have 1 or 2 substituents from the group —OH; NR¹⁶R¹⁷; or 1 substituent from the group consisting of —C(O)OH; and 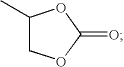
R¹¹ has, independently, the same meaning as R¹;
R¹² has, independently, the same meaning as R²;
R¹³ has, independently, the same meaning as R³, R⁴, R⁵ or R⁶;
R¹⁶, R¹⁷ are, independently of one another, hydrogen or a linear or branched C₁-C₄-alkyl group;
R²² is chosen from the group consisting of C₁-C₄-alkyl groups, acyl groups —C(O)R²³ and the group consisting of ethyleneoxy groups —(CH₂—CH₂O—)_{q}, propyleneoxy groups —(CH(CH₃)—CH₂O—)ᵣ and butyleneoxy groups —(CH₂CH(C₂H₅—O—)ₛ, and mixed alkyleneoxy groups;
R²³ is a C₁-C₁₈-alkyl group;
X and Y in formula I and II are, independently of one another, O or NR²⁴, Z in formula II is N;
R²⁴ is hydrogen or a C₁-C₄-alkyl group;
l, m and n are, independently of one another, 0 or 1;
q is an integer from 1 to 10;
r is an integer from 1 to 10;
s is an integer from 1 to 10;
and where the aliphatic moiety of the compounds of the formula I or II which does not originate from the feed aldehyde in cases where X, Y are ═O and R³, R⁴ or R⁵, R⁶ are ═O must have at least 2 carbon atoms, and in all other cases must have at least 3 carbon atoms.

In this connection, it is preferred if all of the substituents R¹ to R¹³ and the symbols X, Y and Z and l, m, and n have the meanings given above.

Even more preferred are also the compounds of the formulae I and II in which one or more of the symbols X, Y, Z and l, m, and n and also one or more of the substituents R¹ to R¹³ have the following meanings:
R¹ is a linear or branched C₃-C₂₁-alkyl group or a linear or branched C₃-C₂₁-alkenyl group;
R² is —H;
R³, R⁴, R⁵ and R⁶ are, independently of one another, chosen from the group consisting of: —H; —C(O)OH; and C₁-C₃-alkyl groups which may have, at any desired point on the chain, 1 or 2 substituents from the group —OH; —NR¹⁶R¹⁷; —OR²²; or 1 substituent of the type —C(O)OH; or
one of the substituent pairs R³, R⁴ and R⁵, R⁶ is ═O;
R⁷, R⁸, R⁹ and R¹⁰ are, independently of one another, chosen from the group consisting of: —H; —NR¹⁶R¹⁷; —C(O)OH; and C₁-C₃-alkyl groups which may have, at any desired point on the chain, 1 or 2 substituents from the group —OH; NR¹⁶R¹⁷; or 1 substituent of the type —C(O)OH;
R¹¹ has, independently, the same meaning as R¹;
R¹² has, independently, the same meaning as R²;
R¹³ has, independently, the same meaning as R³, R⁴, R⁵ or R⁶;
R¹⁶, R¹⁷ are, independently of one another, hydrogen or a linear or branched C₁-C₄-alkyl group;
R²² is chosen from the group consisting of C₁-C₄-alkyl groups and ethyleneoxy groups —(CH₂—CH₂O—)_{q};
X and Y in formula I and II are, independently of one another, O, S, or NR²⁴, Z in formula II is N;
R²⁴ is hydrogen or a C₁-C₄-alkyl group;
l, m and n are, independently of one another, 0 or 1;
q is an integer from 3 to 8;
and where the aliphatic moiety of the compounds of the formula I or II which does not originate from the feed aldehyde in cases where X, Y are ═O and R³, R⁴ or R⁵, R⁶ are ═O must have at least 2 carbon atoms, and in all other cases must have at least 3 carbon atoms.

In this connection, it is preferred if all of the substituents R¹ to R¹³ and the symbols X, Y and Z and l, m, and n have the meanings given above.

Particular preference is given to the following compounds: 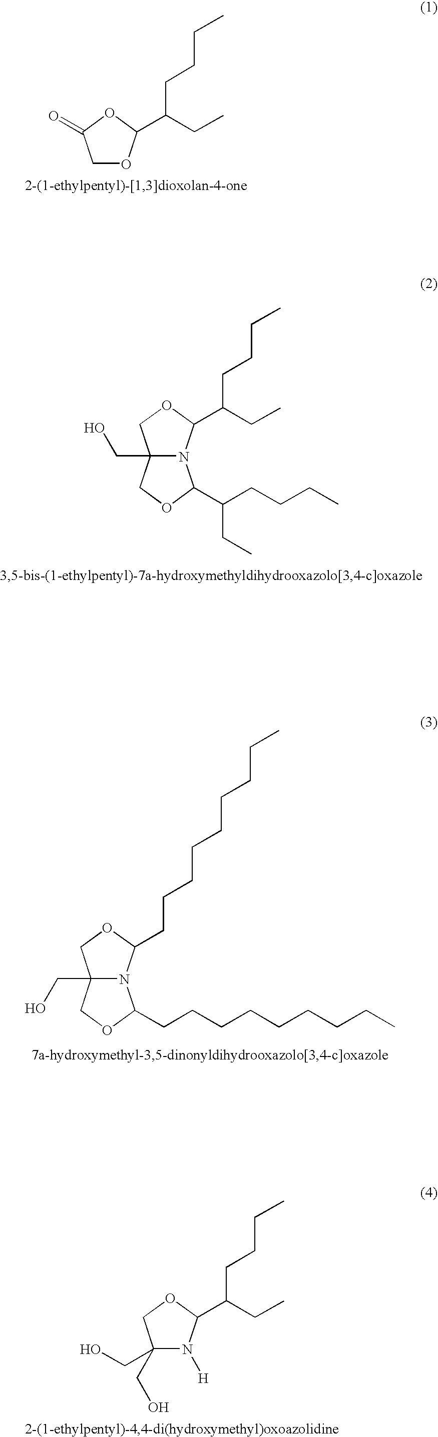 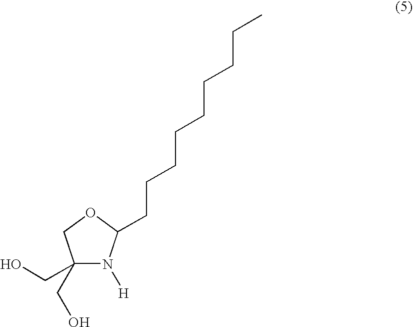
4,4-di(hydroxymethyl)-2-nonyloxazolidine
and
2-(1-propylhexyl)4,4-di(hydroxymethyl)oxazolidine (6);
7a-hydroxymethyl-3,5-di(dodecyl)dihydrooxazolo[3,4-c]oxazole (7);
7a-hydroxymethyl-3,5-di(tetradecyl)dihydrooxazolo[3,4-c]oxazole (8);
7a-hydroxymethyl-3,5-di(undecyl)dihydrooxazolo[3,4-c]oxazole (9);
7a-hydroxymethyl-3,5-di(tridecyl)dihydrooxazolo[3,4-c]oxazole (10);
4,4-di(hydroxymethyl)-2-dodecyloxazolidine (11);
4,4-di(hydroxymethyl)-2-tetradecyloxazolidine (12);
4,4-di(hydroxymethyl)-2-undecyloxazolidine (13);
4,4-di(hydroxymethyl)-2-tridecyloxazolidine (14);
2-(1-propylhexyl)-[1,3]-dioxolan-4-one (15)

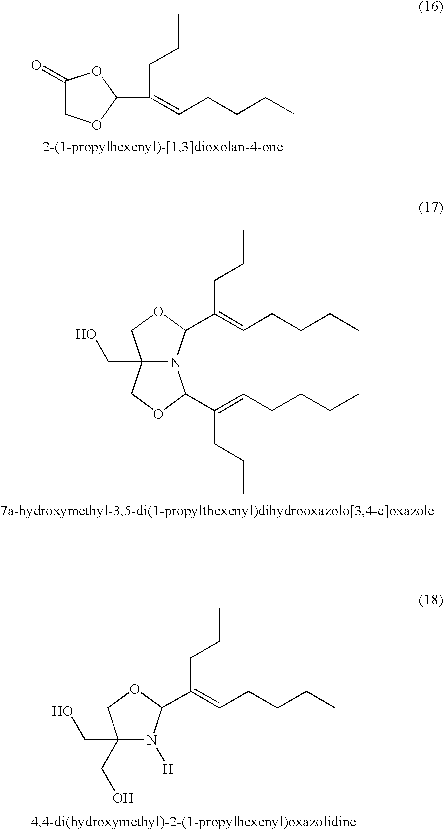

Mixtures of (9) and (10), mixtures of (13) and (14), mixtures of (7) and (8) and mixtures of (11) and (12) are likewise the subject-matter of the most preferred embodiment of the present invention.

A subject-matter of the most preferred embodiment of the present invention are likewise the adducts of the compounds (1) to (18) having 3 to 10 ethylene oxide units and mixtures thereof. In connection with the present invention, the term “mixtures” is understood as meaning both mixtures of adducts of a compound (1) to (18) with varying amounts of added ethylene oxide units, and also mixtures of ethylene oxide adducts of various compounds (1) to (18).

The invention further provides for the use of the compounds of the formulae I and II and the preferred compounds derived therefrom as specified above as cosurfactant.

The compounds of the formulae I and II used according to the invention as cosurfactants are prepared by customary condensation reactions known to the person skilled in the art. The aldehyde and the substance or the substance mixture to be reacted therewith, which is chosen from the abovementioned group of substances, are, optionally in the presence of a suitable acid, reacted together in a suitable solvent, such as, for example, toluene, chloroform or methylene chloride. Suitable acids are the customary Lewis and Brönsted acids known to the person skilled in the art which can be used in gaseous, liquid or solid form. Examples include HCl, sulfuric acid, p-toluenesulfonic acid, p-toluenesulfonic acid pyridinium salt and acidic ion exchangers, for example Amberlyst®15 and Serdolit Red. Alternatively, the starting material to be reacted with the aldehyde can be added to an initial charge of the aldehyde, which may be dissolved in a solvent. This is advantageous particularly in the case of hydroxycarboxylic acids, which can be added as aqueous solution to the reaction mixture. Preferably, the water which forms during the condensation is separated off by distillation. Preference is given to working without solvents. If a solvent is used, the water can be separated off by azeotropic distillation, for example using a water separator.

The substances of the formulae I and II according to the invention to be used as cosurfactants are suitable for use in industrial, institutional or domestic detergents and cleaners, and also in the so-called bodycare sector, namely body-cleansing and bodycare compositions.

Further applications are:
humectants, in particular for the printing industry.
cosmetic, pharmaceutical and crop protection formulations. Suitable crop protection formulations are described, for example in EP-A-0 050 228. Further ingredients customary for crop protection compositions may also be present.
paints, coating compositions, inks, pigment preparations and adhesives in the coating and polymer film industry.
leather fat-liquoring compositions.
formulations for the textile industry, such as leveling agents or formulations for yarn cleaning.
fiber processing and auxiliaries for the paper and pulp industry.
metal processing, such as metal refining and electroplating sector.
food industry.
water treatment and drinking water production.
fermentation.
mineral processing and dust control.
building auxiliaries.
emulsion polymerization and preparation of dispersions.
coolants and lubricants.

The detergents are in solid, liquid, gel or paste form. The materials in solid form include powders and compacts, for example granulates and shaped bodies such as tablets.

The detergents comprise 0.1 to 40% by weight, in particular 0.5 to 30% by weight, very particularly 1 to 20% by weight, based on the total amount of the formulation, of at least one substance of the formulae I and/or II. Further constituents are listed below.

Detergent formulations usually comprise ingredients such as surfactants, builders, fragrances and dyes, complexing agents, polymers and other ingredients. Typical formulations are described, for example, in WO 01/32820. Further ingredients suitable for various applications are described in EP-A-0 620 270, WO 95/27034, EP-A-0 681 865, EP-A-0 616 026, EP-A-0 616 028, DE-A-42 37 178 and U.S. Pat. No. 5,340,495, for example.

For the purposes of this invention, detergents are generally used for the washing of materials of greater or lesser flexibility, preferably those which contain or consist of natural, synthetic or semisynthetic fiber materials and which consequently usually have at least partially a textile character. The materials which contain or consist of fibers can, in principle, be in any form which exists in use or for the preparation and processing. For example, fibers may be unarranged in the form of staple or aggregate, arranged in the form of threads, yarns, twines, or in the form of fabrics, such as nonwovens, loden materials or felt, wovens, knits in all conceivable types of weave.

These may be raw fibers or fibers in any stages of processing and may be natural protein or cellulose fibers, such as wool, silk, cotton, sisal, hemp, coconut fibers or synthetic fibers, such as, for example, polyester, polyamide or polyacrylonitrile fibers.

Detergents comprising cosurfactants according to the invention can also be used for cleaning fiber-containing materials, such as e.g. backed carpets with cut or uncut pile.

The compositions of the detergents are preferably adapted to the different purposes, as is familiar to the person skilled in the art from the prior art. For this purpose, all auxiliaries and additives corresponding to the purpose and known from the prior art can be added to the detergents.

In addition to the cosurfactants according to the invention, the following may, for example, be present in detergents:
builders and cobuilders, such as polyphosphates, zeolites, polycarboxylates, phosphonates or complexing agents
ionic surfactants, such as alcohol sulfates/ether sulfates, alkylbenzenesulfonates, α-olefinsulfonates and other alcohol sulfates/ether sulfates
nonionic surfactants, alcohol alkoxyates such as alkylamine alkoxylates, alkyl polyglucosides
optical brighteners
color transfer inhibitors, such as polyvinylpyrrolidone of molar masses 8000 to 70 000, vinylimidazole/vinylpyrrolidone copolymers with a molar ratio of the monomers of from 1:10 to 2:1 and molar masses of from 8000 to 70 000, and poly-4-vinylpyridine N-oxides with molar masses of from 8000 to 70 000
extenders, such as sodium sulfate or magnesium sulfate
soil release agent
incrustation inhibitors
bleaching systems, comprising bleach, such as perborate, percarbonate and bleach activators, such as tetraacetylethylenediamine, and also bleach stabilizers
perfume (oils)
foam suppressors, such as silicone oils
enzymes, such as amylases, lipases, cellulases, proteases
alkali metal donors, such as soluble alkali metal silicates, e.g. pentasodium methasilicate, sodium carbonate.

Solvents, such as ethanol, isopropanol, 1,2-propylene glycol, butyl glycol etc., can, for example, additionally be used in liquid detergents.

In tablet detergents, it is additionally possible to use tableting auxiliaries, such as polyethylene glycols with molar masses of more than 1000 g/mol, polymer dispersions, and tablet disintegrants, such as cellulose derivatives, crosslinked polyvinylpyrrolidone, crosslinked polyacrylates or combinations of acids, such as citric acid and sodium bicarbonate. A detailed list of possible ingredients is given below.

In some cases, it may be expedient to combine the cosurfactants used according to the invention with other cosurfactants or with amphoteric surfactants, such as, for example, alkylamine oxides, or betaines.

Another class of nonionic surfactants are alkyl polyglucosides having 6 to 22, preferably 10 to 18, carbon atoms in the alkyl chain. These compounds generally contain 1 to 20, preferably 1.1 to 5, glucoside units.

Another class of nonionic surfactants are N-alkylglucamides of the structures 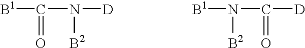
where B¹ is a C₆- to C₂₂-alkyl, B² is hydrogen or C₁- to C₄-alkyl and D is a polyhydroxyalkyl radical having 5 to 12 carbon atoms and at least 3 hydroxyl groups. Preferably, B¹ is C₁₀- to C₁₈-alkyl, B² is CH₃ and D is a C₅- or C₆-radical. For example, such compounds are obtained by the acylation of reductively aminated sugars with acid chlorides of C₁₀- to C₁₈-carboxylic acids.

Further suitable nonionic surfactants are the terminally capped fatty acid amide alkoxylates, known from WO-A 95/11225, of the formula
R¹—CO—NH—(CH₂)_{y}—O—(A¹O)ₓ—R²
in which
R¹ is a C₅- to C₂₁-alkyl or alkenyl radical,
R² is a C₁- to C₄-alkyl group,
A¹ is C₂- to C₄-alkylene,
y is the number 2 or 3 and
x has a value from 1 to 6.

Examples of such compounds are the reaction products of n-butyltriglycolamine of the formula H₂N—(CH₂—CH₂—O)₃—C₄H₉ with methyl dodecanoate or the reaction products of ethyltetraglycolamine of the formula H₂N—(CH₂—CH₂—O)₄—C₂H₅ with a standard commercial mixture of saturated C₈- to C₁₈-fatty acid methyl esters.

Further suitable nonionic surfactants are also block copolymers of ethylene oxide, propylene oxide and/or butylene oxide (Pluronic® and Tetronic® brands from BASF), polyhydroxy or polyalkoxy fatty acid derivatives, such as polyhydroxy fatty acid amides, N-alkoxy- or N-aryloxypolyhydroxy fatty acid amides, fatty acid amide ethoxylates, in particular terminally capped ones, and fatty acid alkanolamide alkoxylates.

The additional nonionic surfactants are present in the detergents comprising the cosurfactants used in accordance with the invention preferably in an amount of from 0.01 to 30% by weight, in particular 0.1 to 25% by weight, especially 0.5 to 20% by weight.

It is also possible to use individual nonionic surfactants or a combination of different nonionic surfactants. The nonionic surfactants used may come from only one class, in particular only alkoxylated C₈- to C₂₂-alcohols, or surfactant mixtures from different classes can be used.

Suitable anionic surfactants are, for example, fatty alcohol sulfates of fatty alcohols having 8 to 22, preferably 10 to 18, carbon atoms, C₁₂-C₁₈-alcohol sulfates, lauryl sulfate, cetyl sulfate, myristyl sulfate, palmityl sulfate, stearyl sulfate and tallow fatty alcohol sulfate.

Further suitable anionic surfactants are sulfated ethoxylated C₈- to C₂₂-alcohols (alkyl ether sulfates) or soluble salts thereof. Compounds of this type are prepared, for example, by firstly alkoxylating a C₈- to C₂₂-, preferably a C₁₀- to C₁₈-alcohol, e.g. a fatty alcohol, and then sulfating the alkoxylation product. For the alkoxylation, preference is given to using ethylene oxide, 1 to 50 mol, preferably 1 to 20 mol, of ethylene oxide being used per mole of alcohol. The alkoxylation of the alcohols can, however, also be carried out with propylene oxide on its own and optionally butylene oxide. Furthermore, also suitable are those alkylated C₈- to C₂₂-alcohols which contain ethylene oxide and propylene oxide or ethylene oxide and butylene oxide or ethylene oxide and propylene oxide and butylene oxide. The alkoxylated C₈- to C₂₂-alcohols can contain the ethylene oxide, propylene oxide and butylene oxide units in the form of blocks or in random distribution. Depending on the nature of the alkoxylation catalyst, alkyl ether sulfates can be obtained with a broad or narrow alkylene oxide homolog distribution.

Further suitable anionic surfactants are alkanesulfonates, such as C₈- to C₂₄-, preferably C₁₀- to C₁₈-alkanesulfonates, and soaps, such as, for example, the Na and K salts of saturated and/or unsaturated C₈- to C₂₄-carboxylic acids.

Further suitable anionic surfactants are linear C₈- to C₂₀-alkylbenzenesulfonates (“LAS”), preferably linear C₉- to C₁₃-alkylbenzenesulfonates and -alkyltoluenesulfonates.

Further suitable anionic surfactants are also C₈- to C₂₄-olefinsulfonates and -disulfonates, which may also represent mixtures of alklene- and hydroxyalkanesulfonates or-disulfonates, alkyl ester sulfonates, sulfonated polycarboxylic acids, alkylglycerol sulfonates, fatty acid glycerol ester sulfonates, alkylphenol polyglycol ether sulfates, paraffinsulfonates having about 20 to about 50 carbon atoms (based on paraffin or paraffin mixtures obtained from natural sources), alkyl phosphates, acyl isethionates, acyl taurates, acyl methyltaurates, alkylsuccinic acids, alkenylsuccinic acids or half-esters or half-amides thereof, alkylsulfosuccinic acids or amides thereof, mono- and diesters of sulfosuccinic acids, acyl sarcosinates, sulfated alkyl polyglucosides, alkyl polyglycol carboxylates and hydroxyalkyl sarcosinates.

The anionic surfactants are preferably added to the detergent in the form of salts. Suitable cations in these salts are alkali metal ions, such as sodium, potassium and lithium and ammonium salts, such as, e.g. hydroxyethylammonium, di(hydroxyethyl)ammonium and tri(hydroxyethyl)ammonium salts.

The anionic surfactants are present in the detergents comprising the cosurfactants according to the invention preferably in an amount of up to 30% by weight, for example from 0.1 to 30% by weight, especially 1 to 25% by weight, in particular 3 to 10% by weight. If C₉- to C₂₀ linear alkyl-benzenesulfonates (LAS) are co-used, these are usually employed in an amount up to 15% by weight, in particular up to 10% by weight.

It is possible to use individual anionic surfactants or a combination of different anionic surfactants. The anionic surfactants used may be from only one class, for example only fatty alcohol sulfates or only alkylbenzenesulfonates, although it is also possible to use surfactant mixtures from different classes, e.g. a mixture of fatty alcohol sulfates and alkylbenzenesulfonates.

In addition, the surfactant mixtures comprising the cosurfactants to be used according to the invention can be combined with cationic surfactants, customarily in an amount up to 25% by weight, preferably 1 to 15% by weight, for example C₈- to C₁₆-dialkyldimethylammonium salts, dialkoxydimethylammonium salts or imidazolinium salts with a long-chain alkyl radical; and/or with amphoteric surfactants, customarily in an amount up to 15% by weight, preferably 1 to 10% by weight, for example derivatives of secondary or tertiary amines, such as, e.g. C₆-C₁₈-alkylbetaines or C₆-C₁₅-alkylsulfobetaines or alkylamidobetaines or amine oxides, such as alkyldimethylamine oxides.

It is also possible to use cationic surfactants as are described in WO 99/19435.

The mixtures comprising the cosurfactants to be used in accordance with the invention are usually combined with builders (sequestering agents), such as, for example, polyphosphates, polycarboxylates, phosphonates, complexing agents, e.g. methylglycinediacetic acid and salts thereof, nitrilotriacetic acid and salts thereof, ethylenediaminetetraacetic acid and salts thereof, and optionally with cobuilders.

Individual builder substances which are highly suitable for the combination with mixtures comprising the cosurfactants to be used in accordance with the invention may be listed below:

Suitable inorganic builders are primarily crystalline or amorphous alumosilicates having ion-exchanging properties, such as, in particular, zeolites. Various types of zeolites are suitable, in particular zeolites A, X, B, P, MAP and HS in their Na form or in forms in which Na is partially replaced by other cations, such as Li, K, Ca, Mg or ammonium. Suitable zeolites are described, for example, in U.S. Pat. No. 4,604,224.

Examples of crystalline silicates which are suitable as builders are disilicates or phyllosilicates, e.g. δ-Na₂Si₂O₅ or β-Na₂Si₂O₅. The silicates can be used in the form of their alkali metal, alkaline earth metal or ammonium salts, preferably as Na, Li and Mg silicates. Amorphous silicates, such as, for example, sodium metasilicate, which has a polymeric structure, or amorphous disilicate can likewise be used.

Suitable carbonate-based inorganic builder substances are carbonates and hydrogencarbonates. These can be used in the form of their alkali metal, alkaline earth metal or ammonium salts. Preference is given to using Na, Li and Mg carbonates or hydrogencarbonates, in particular sodium carbonate and/or sodium hydrogencarbonate.

Customary phosphates used as inorganic builders are alkali metal orthophosphates and/or polyphosphates, such as, for example, pentasodium triphosphate.

Said builder components can be used individually or in mixtures with one another.

In addition, in many cases, it is expedient to add cobuilders to the detergents comprising the cosurfactants to be used in accordance with the invention. Examples of suitable substances are listed below:

In a preferred embodiment, the detergents comprising the cosurfactants to be used in accordance with the invention comprise, in addition to the inorganic builders, 0.05 to 20% by weight, in particular 1 to 10% by weight, of organic cobuilders in the form of low molecular weight, oligomeric or polymeric carboxylic acids, in particular polycarboxylic acids, or phosphonic acids or salts thereof, in particular Na or K salts.

Low molecular weight carboxylic acids or phosphonic acids suitable as organic cobuilders are, for example, phosphonic acids, such as, for example, 1-hydroxyethane-1,1-diphosphonic acid, amino-tris(methylenephosphonic acid), ethylenediaminetetra-(methylenephosphonic acid), hexamethylenediaminetetra(methylenephosphonic acid) and diethylenetriaminepenta(methylenephosphonic acid);
C₄- to C₂₀-di-, -tri- and -tetracarboxylic acids, such as, for example, succinic acid, propanetricarboxylic acid, butanetetracarboxylic acid, cyclopentanetetracarboxylic acid and alkyl- and alkenylsuccinic acids having C₂- to C₁₆-alkyl- or -alkenyl radicals;
C₄- to C₂₀-hydroxycarboxylic acids, such as, for example, malic acid, tartaric acid, gluconic acid, glutaric acid, citric acid, lactobionic acid and sucrose mono-, di- and tricarboxylic acid;
aminopolycarboxylic acids, such as, for example, nitrilotriacetic acid, β-alaninediacetic acid, ethylenediaminetetraacetic acid, serinediacetic acid, isoserinediacetic acid, alkylethylenediaminetriacetates, N,N-bis(carboxymethyl)glutamic acid, ethylenediamine-disuccinic acid and N-(2-hydroxyethyl)iminodiacetic acid, methyl- and ethylglycinediacetic acid.

Examples of oligomeric or polymeric carboxylic acids which are suitable as organic cobuilders are:

oligomaleic acids, as are described, for example, in EP-A 451508 and EP-A 396303;

co- and terpolymers of unsaturated C₄- to C₈-dicarboxylic acids, the copolymerized comonomers being monoethylenically unsaturated monomers from group (i), given below, in amounts of up to 95% by weight, from group (ii) in amounts of up to 60% by weight and from group (iii) in amounts of up to 20% by weight.

Examples of unsaturated C₄- to C₈-dicarboxylic acids in this context are maleic acid, fumaric acid, itaconic acid and citraconic acid. Preference is given to maleic acid.

Group (i) includes monoethylenically unsaturated C₃-C₈-monocarboxylic acids, such as, for example, acrylic acid, methacrylic acid, crotonic acid and vinylacetic acid. From group (i), preference is given to using acrylic acid and methacrylic acid.

Group (ii) includes monoethylenically unsaturated C₂- to C₂₂-olefins, vinyl alkyl ethers having C₁- to C₈-alkyl groups, styrene, vinyl esters of C₁- to C₈-carboxylic acids, (meth)acrylamide and vinylpyrrolidone. From group (ii), preference is given to using C₂- to C₆-olefins, vinyl alkyl ethers having C₁- to C₄-alkyl groups, vinyl acetate and vinyl propionate.

If the polymers of group (ii) contain copolymerized vinyl esters, some or all of the latter can also be present in hydrolyzed form to give vinyl alcohol structural units. Suitable co- and terpolymers are known, for example, from U.S. Pat. No. 3,887,806 and DE-A 4313909.

Group (iii) includes (meth)acrylic esters of C₁- to C₈-alcohols, (meth)acrylonitrile, (meth)acrylamides of C₁- to C₈-amines, N-vinylformamide and N-vinylimidazole.

Also suitable as organic cobuilders are homopolymers of monoethylenically unsaturated C₃-C₈-monocarboxylic acids, such as, for example, acrylic acid, methacrylic acid, crotonic acid and vinylacetic acid, in particular acrylic acid and methacrylic acid;

copolymers of dicarboxylic acids, such as, for example, copolymers of maleic acid and acrylic acid in the weight ratio 10:90 to 95:5, particularly preferably those in the weight ratio 30:70 to 90:10 with molar masses of from 1000 to 150 000;

terpolymers of maleic acid, acrylic acid and a vinyl ester of a C₁-C₃-carboxylic acid in the weight ratio 10 (maleic acid):90 (acrylic acid+vinyl ester) to 95 (maleic acid):10 (acrylic acid+vinyl ester), where the weight ratio of acrylic acid to the vinyl ester can vary within the range from 30:70 to 70:30;

copolymers of maleic acid with C₂-C₈-olefins in the molar ratio 40:60 to 80:20, copolymers of maleic acid with ethylene, propylene or isobutene in the molar ratio 50:50 being particularly preferred.

Graft polymers of unsaturated carboxylic acids onto low molecular weight carbohydrates or hydrogenated carbohydrates, cf. U.S. Pat. No. 5,227,446, DE-A 4415623 and DE-A 4313909, are likewise suitable as organic cobuilders.

Examples of suitable unsaturated carboxylic acids in this context are maleic acid, fumaric acid, itaconic acid, citraconic acid, acrylic acid, methacrylic acid, crotonic acid and vinylacetic acid, and also mixtures of acrylic acid and maleic acid which are grafted on in amounts of from 40 to 95% by weight, based on the component to be grafted.

For modification, it is additionally possible for up to 30% by weight, based on the component to be grafted, of further monoethylenically unsaturated monomers to be present in copolymerized form. Suitable modifying monomers are the abovementioned monomers of groups (ii) and (iii).

Suitable graft bases are degraded polysaccharides, such as, for example, acidically or enzymatically degraded starches, inulins or cellulose, protein hydrolyzates and reduced (hydrogenated or reductively aminated) degraded polysaccharides, such as, for example, mannitol, sorbitol, aminosorbitol and N-alkylglucamine, and also polyalkylene glycols with molar masses up to M_{w}=5000, such as, for example, polyethylene glycols, ethylene oxide/propylene oxide or ethylene oxide/butylene oxide or ethylene oxide/propylene oxide/butylene oxide block copolymers and alkoxylated mono- or polyhydric C₁- to C₂₂-alcohols (cf. U.S. Pat. No. 5,756,456).

Polyglyoxylic acids suitable as organic cobuilders are described, for example, in EP-B-001004, U.S. Pat. No. 5,399,286, DE-A-4106355 and EP-A-656914. The end groups of the polyglyoxylic acids may have different structures.

Polyamidocarboxylic acids and modified polyamidocarboxylic acids suitable as organic cobuilders are known, for example, from EP-A-454126, EP-B-511037, WO-A-94/01486 and EP-A-581452.

In particular, polyaspartic acids or cocondensates of aspartic acid with further amino acids, C₄- to C₂₅-mono- or -dicarboxylic acids and/or C₄- to C₂₅-mono- or -diamines are also used as organic cobuilders. Particular preference is given to using polyaspartic acids which have been prepared in phosphorus-containing acids and modified with C₆- to C₂₂-mono- or -dicarboxylic acids or with C₆- to C₂₂-mono- or -diamines.

Also suitable as organic cobuilders are iminodisuccinic acid, oxydisuccinic acid, aminopolycarboxylates, alkylpolyaminocarboxylates, aminopolyalkylenephosphonates, polyglutamates, hydrophobically modified citric acid, such as, for example, agaric acid, poly-α-hydroxyacrylic acid, N-acylethylenediaminetriacetates, such as lauroyl ethylenediaminetriacetate and alkylamides of ethylenediaminetetraacetic acid, such as EDTA-tallow amide.

Furthermore, it is also possible to use oxidized starches as organic cobuilders.

Further suitable (co)builders are described in WO 99/19435.

In a further preferred embodiment, the detergents comprising the cosurfactants to be used in accordance with the invention additionally comprise, in particular in addition to the inorganic builders, the anionic surfactants and/or the nonionic surfactants, 0.5 to 20% by weight, in particular 1 to 10% by weight, of glycine-N,N-diacetic acid derivatives, as described in WO 97/19159.

It is also frequently expedient to add bleaching systems, consisting of bleaches, such as, for example, perborate, percarbonate, and optionally bleach activators, such as, for example, tetraacetylethylenediamine, +bleach stabilizers and optionally bleach catalysts to the detergents comprising the cosurfactants to be used in accordance with the invention.

In these cases, the detergents comprising the cosurfactants to be used in accordance with the invention additionally comprise 0.5 to 30% by weight, in particular 5 to 27% by weight, especially 10 to 23% by weight, of bleaches in the form of percarboxylic acids, e.g. diperoxododecanedicarboxylic acid, phthalimidopercaproic acid, or monoperoxophthalic acid or -terephthalic acid, adducts of hydrogen peroxide with inorganic salts, e.g. sodium perborate monohydrate, sodium perborate tetrahydrate, sodium carbonate perhydrate or sodium phosphate perhydrate, adducts of hydrogen peroxide with organic compounds, e.g. urea perhydrate, or of inorganic peroxo salts, e.g. alkali metal persulfates or peroxodisulfates, optionally in combination with 0 to 15% by weight, preferably 0.1 to 15% by weight, in particular 0.5 to 8% by weight, of bleach activators.

Suitable bleach activators are:
polyacylated sugars, e.g. pentaacetylglucose;
acyloxybenzenesulfonic acids and alkali metal and alkaline earth metal salts thereof, e.g. sodium p-nonanoyloxybenzenesulfonate or sodium p-benzoyloxybenzene-sulfonate;
N,N-diacylated and N,N,N′,N′-tetraacylated amines, e.g. N,N,N′,N′-tetraacetyl-methylenediamine and -ethylenediamine (TAED), N,N-diacetylaniline, N,N-diacetyl-p-toluidine or 1,3-diacylated hydantoins, such as 1,3-diacetyl-5,5-dimethylhydantoin;
N-alkyl-N-sulfonylcarbonamides, e.g. N-methyl-N-mesylacetamide or N-methyl-N-mesylbenzamide;
N-acylated cyclic hydrazides, acylated triazoles or urazoles, e.g. monoacetylmaleic hydrazide;
O,N,N-trisubstituted hydroxylamines, e.g. O-benzoyl-N,N-succinylhydroxylamine, O-acetyl-N,N-succinylhydroxylamine or O,N,N-triacetylhydroxylamine;
N,N′-diacylsulfurylamides, e.g. N,N′-dimethyl-N,N′-diacetylsulfurylamide or N,N′-diethyl-N,N′-dipropionylsulfurylamide;
acylated lactams, such as, for example, acetylcaprolactam, octanoylcaprolactam, benzoylcaprolactam or carbonylbiscaprolactam;
anthranil derivatives, such as, for example, 2-methylanthranil or 2-phenylanthranil;
triacyl cyanurates, e.g. triacetyl cyanurate or tribenzoyl cyanurate;
oxime esters and bisoxime esters, such as, for example, O-acetylacetone oxime or bisisopropyliminocarbonate;
carboxylic anhydrides, e.g. acetic anhydride, benzoic anhydride, m-chlorobenzoic anhydride or phthalic anhydride;
enol esters, such as, for example, isopropenyl acetate;
1,3-diacyl-4,5-diacyloxyimidazolines, e.g. 1,3-diacetyl-4,5-diacetoxyimidazoline;
tetraacetylglycoluril and tetrapropionylglycoluril;
diacylated 2,5-diketopiperazines, e.g. 1,4-diacetyl-2,5-diketopiperazine;
ammonium-substituted nitriles, such as, for example, N-methylmorpholinium acetonitrile methylsulfate;
acylation products of propylenediurea and 2,2-dimethylpropylenediurea, e.g. tetraacetylpropylenediurea;
α-acyloxypolyacylmalonamides, e.g. α-acetoxy-N,N′-diacetylmalonamide;
diacyldioxohexahydro-1,3,5-triazines, e.g. 1,5-diacetyl-2,4-dioxohexahydro-1,3,5-triazine;
benz-(4H)1,3-oxazin-4-ones having alkyl radicals, e.g. methyl, or aromatic radicals, e.g. phenyl, in the 2-position;
cationic nitriles, as described in DE-A-101 48 577.

The described bleaching system comprising bleaches and bleach activators can optionally also comprise bleach catalysts. Examples of suitable bleach catalysts are quaternized imines and sulfonimines, which are described, for example, in U.S. Pat. No. 5,360,569 and EP-A 453 003. Particularly effective bleach catalysts are manganese complexes, which are described, for example, in WO-A 94/21777. Where used, such compounds are incorporated into the detergents in amounts of at most up to 1.5% by weight, in particular up to 0.5% by weight, and in the case of very active manganese complexes, in amounts up to 0.1% by weight. Further suitable bleach catalysts are described in WO 99/19435.

Further bleaching systems based on arylimidoperalkanoic acids which can be used are described in EP-A-0 325 288 and EP-A-0 490 409.

Bleach Stabilizer

These are additives which are able to absorb, bind or complex traces of heavy metals. Examples of additives with a bleach-stabilizing action which can be used according to the invention are polyanionic compounds, such as polyphosphates, polycarboxylates, polyhydroxypolycarboxylates, soluble silicates as completely or partially neutralized alkali metal or alkaline earth metal salts, in particular as neutral Na or Mg salts which are relatively weak bleach stabilizers. Strong bleach stabilizers which can be used according to the invention are, for example, complexing agents, such as ethylenediamine tetraacetate (EDTA), nitrilotriacetic acid (NTA), methylglycinediacetic acid (MGDA), β-alaninediacetic acid (ADA), ethylenediamine N,N′-disuccinate (EDDS) and phosphonates, such as ethylenediaminetetramethylenephosphonate, diethylenetriamine-pentamethylenephosphonate or hydroxyethylidene-1,1-diphosphonic acid in the form of the acids or as partially or completely neutralized alkali metal salts. The complexing agents are preferably used in the form of their Na salts.

As well as the described bleaching system comprising bleaches, bleach activators and optionally bleach catalysts, the use of systems with enzymatic peroxide release or of photoactivated bleaching systems is also possible for the detergents comprising the cosurfactants to be used in accordance with the invention, see e.g. U.S. Pat. No. 4,033,718.

For a number of uses, it is expedient for the detergents comprising the cosurfactants to be used in accordance with the invention to comprise enzymes. Enzymes which are preferably used in detergents are proteases, amylases, lipases and cellulases. Preferred amounts of the enzymes are from 0.1 to 1.5% by weight, particularly preferably 0.2 to 1.0% by weight, of the formulated enzyme. Examples of suitable proteases are Savinase and Esperase. A suitable lipase is e.g. Lipolase. A suitable cellulase is e.g. Celluzym. The use of peroxidases for activating the bleaching system is also possible. It is possible to use individual enzymes or a combination of different enzymes. Where appropriate, the detergent comprising the cosurfactants to be used in accordance with the invention can also comprise enzyme stabilizers, e.g. calcium propionate, sodium formate or boric acids or salts thereof, and/or antioxidants.

The constituents of detergents are known in principle to the person skilled in the art. The lists, above and below, of suitable constituents give merely an illustrative selection of the known suitable constituents.

In addition to the main components stated hitherto, the detergents comprising the cosurfactants to be used in accordance with the invention can also comprise the following further customary additives in the amounts customary for this purpose:

Known dispersants, such as naphthalenesulfonic acid condensates or polycarboxylates, soil-carrying agents, soil release agents, such as polyether esters, incrustation inhibitors, pH-regulating compounds, such as alkalis or alkali donors (NaOH, KOH, pentasodium metasilicate, sodium carbonate) or acids (hydrochloric acid, phosphoric acid, amidosulfuric acid, citric acid), buffer systems, such as acetate or phosphate buffer, ion exchangers, perfume, dyes, graying inhibitors, optical (fluorescent) brighteners, color-transfer inhibitors, such as, for example, polyvinylpyrrolidone, biocides, such as isothiazolinones or 2-bromo-2-nitro-1,3-propanediol, hydrotropic compounds as solubility promoters or solubilizers, such as cumenesulfonates, toluenesulfonates, short-chain fatty acids, urea, alcohols or phosphoric alkyl/aryl esters, foam regulators for stabilizing or suppressing foam, e.g. silicone oils, skin and corrosion protectants, disinfecting compounds or systems, such as, for example, those which release chlorine or hypochlorous acid, such as dichloroisocyanurate or which contain iodine, thickeners and extenders and formulating agents.

Graying Inhibitors and Soil Release Polymers

Suitable soil release polymers and/or graying inhibitors for detergents are for example:

polyesters of polyethylene oxides with ethylene glycol and/or propylene glycol and aromatic dicarboxylic acids or aromatic and aliphatic dicarboxylic acids;

polyesters of unilaterally terminally capped polyethylene oxides with di- and/or polyhydric alcohols and dicarboxylic acid.

Such polyesters are known, for example from U.S. Pat. No. 3,557,039, GB-A 1 154 730, EP-A-185 427, EP-A-241 984, EP-A-241 985, EP-A-272 033 and U.S. Pat. No. 5,142,020.

Further suitable soil release polymers are amphiphilic graft or copolymers of vinyl and/or acrylic esters onto polyalkylene oxides (cf U.S. Pat. No. 4,746,456, U.S. Pat. No. 4,846,995, DE-A-37 11 299, U.S. Pat. No. 4,904,408, U.S. Pat. No. 4,846,994 and U.S. Pat. No. 4,849,126) or modified celluloses, such as, for example, methylcellulose, hydroxypropylcellulose or carboxymethylcellulose.

Color Transfer Inhibitors

The color transfer inhibitors used are, for example, homo- and copolymers of vinylpyrrolidone, of vinylimidazole, of vinyloxazolidone and of 4-vinylpyridine N-oxide having molar masses of from 15 000 to 100 000, and crosslinked finely divided polymers based on these monomers. The use mentioned here of such polymers is known, cf. DE-B-22 32 353, DE-A-28 14 287, DE-A-28 14 329 and DE-A-43 16 023.

Suitable polyvinylpyridinebetaines are described, for example in Tai, Formulating Detergents and Personal Care Products, AOCS Press, 2000, page 113.

In addition to the use in detergents and cleaners for domestic textile washing, the detergent compositions which can be used according to the invention can also be used in the field of commercial textile washing and of commercial cleaning. In this field of use, peracetic acid is usually used as bleach, and is added to the wash liquor as an aqueous solution.

Use in Textile Detergents

A typical pulverulent or granular heavy-duty detergent according to the invention may, for example, have the following composition:
0.5 to 50% by weight, preferably 5 to 30% by weight, of at least one anionic and/or nonionic surfactant, including the cosurfactants according to the invention,
0.5 to 60% by weight, preferably 15 to 40% by weight, of at least one inorganic builder,
0 to 20% by weight, preferably 0.5 to 8% by weight, of at least one organic cobuilder,
2 to 35% by weight, preferably 5 to 30% by weight, of an inorganic bleach,
0.1 to 20% by weight, preferably 0.5 to 10% by weight, of a bleach activator, optionally in a mixture with further bleach activators,
0 to 1% by weight, preferably up to at most 0.5% by weight, of a bleach catalyst,
0 to 5% by weight, preferably 0 to 2.5%, of a polymeric color transfer inhibitor,
0 to 1.5% by weight, preferably 0.1 to 1.0% by weight, of protease,
0 to 1.5% by weight, preferably 0.1 to 1.0% by weight, of lipase,
0 to 1.5% by weight, preferably 0.2 to 1.0% by weight, of a soil release polymer,
   ad 100% of customary auxiliaries and adjuncts and water.

Inorganic builders preferably used in detergents are sodium carbonate, sodium hydrogencarbonate, zeolite A and P, and amorphous and crystalline Na silicates, and also phyllosilicates.

Organic cobuilders preferably used in detergents are acrylic acid/maleic acid copolymers, acrylic acid/maleic acid/vinyl ester terpolymers and citric acid.

Inorganic bleaches preferably used in detergents are sodium perborate and sodium carbonate perhydrate.

Anionic surfactants preferably used in detergents are linear and slightly branched alkylbenzenesulfonates (LAS), fatty alcohol sulfates/ether sulfates and soaps.

Enzymes preferably used in detergents are protease, lipase, amylase and cellulase. For the commercially available enzymes, amounts of from 0.05 to 2.0% by weight, preferably 0.2 to 1.5% by weight, of the formulated enzyme, are generally added to the detergent. Suitable proteases are, for example, Savinase, Desazym and Esperase. A suitable lipase is, for example, Lipolase. A suitable cellulase is, for example, Celluzym.

Soil release polymers and graying inhibitors preferably used in detergents are graft polymers of vinyl acetate onto polyethylene oxide of molar mass 2500-8000 in the weight ratio 1.2:1 to 3.0:1, polyethylene terephthalates/oxyethylene terephthalates of molar mass 3000 to 25 000 from polyethylene oxides of molar mass 750 to 5000 with terephthalic acid and ethylene oxide and a molar ratio of polyethylene terephthalate to polyoxyethylene terephthalate of from 8:1 to 1:1, and block polycondensates according to DE-A-44 03 866.

Color transfer inhibitors preferably used in detergents are soluble NVP homopolymers and/or vinylpyrrolidone and vinylimidazole copolymers with molar masses greater than 5000.

The detergents are often in solid, pulverulent form, in which case they usually additionally comprise customary extenders, which give them good flowability, dosability and solubility and which prevent caking and dusting, such as sodium sulfate or magnesium sulfate.

The pulverulent or granular detergents according to the invention can comprise up to 60% by weight of inorganic extenders. However, the detergents according to the invention preferably have a low content of extenders and comprise only up to 20% by weight, particularly preferably only up to 8% by weight, of extenders.

Detergents comprising the cosurfactants to be used in accordance with the invention can have various bulk densities in the range from 300 to 1200, in particular 500 to 950 g/l. Modern compact detergents usually have high bulk densities and are granular in structure. Compact or ultracompact detergents and extrudates have a bulk density of >600 g/l. These are becoming more important.

If they are to be used in liquid form, they may be in the form of aqueous microemulsions, emulsions or solutions. In liquid detergents, solvents such as ethanol, isopropanol, 1,2-propylene glycol or butyl glycol can additionally be used.

In the case of gel detergents thickeners, such as, for example, polysaccharides and/or weakly crosslinked polycarboxylates (for example Carbopol® from Goodrich) can additionally be used.

In the case of tablet detergents, tableting auxiliaries, such as, for example, polyethylene glycols with molar masses of >1000 g/mol, polymer dispersions, and tablet disintegrants such as cellulose derivatives, crosslinked polyvinylpyrrolidone, crosslinked polyacrylates or combinations of acids, e.g. citric acid+sodium bicarbonate, to name but a few, are additionally required.

The present invention further provides for the use of the mixtures in the preparation of detergents.

In connection with the present invention, the term “cleaners” is generally understood as meaning formulations which are used for cleaning hard surfaces. They are in liquid, gel, paste or solid form. Materials which are a type of solid form include powders and compacts, such as, for example granulates and shaped bodies, for example tablets. Examples include hand dishwashing detergents, machine dishwashing detergents, metal degreasers, glass cleaners, floor cleaners, all-purpose cleaners, high-pressure cleaners, alkaline cleaners, acidic cleaners, spray degreasers, dairy cleaners, upholstery cleaners, plastics cleaners and bath cleaners. They comprise 0.01 to 40% by weight, preferably 0.1 to 25% by weight, based on the total formulation, of at least one substance of the formulae I and/or II. Further constituents are detailed below.
ionic surfactants, such as, for example, alcohol sulfate/ether sulfates, alkylbenzene sulfonates, α-olefinsulfonates, sulfosuccinates, as described above under “detergents”.
nonionic surfactants, such as, for example, alcohol alkoxylates, alkylamine alkoxylates, alkylamide ethoxylates, alkyl polyglucosides, as described above under “detergents”.
amphoteric surfactants, such as, for example, alkylamine oxides, betaines, as described above under “detergents”.
builders, such as, for example, polyphosphates, polycarboxylates, phosphonates, complexing agents, e.g. methylglycine diacetic acid and salts thereof, nitrilotriacetic acid and salts thereof, ethylenediamine tetraacetic acid and salts thereof, as described above under “detergents”.
dispersants, such as, for example, naphthalenesulfonic acid condensates, polycarboxylates, as described above under “detergents”.
pH-regulating compounds, such as, for example, alkalis (NaOH, KOH, pentasodium metasilicate) or acids (hydrochloric acid, phosphoric acid, amidosulfuric acid, citric acid)
enzymes, such as, for example lipases, amylases, proteases
perfume
dyes
biocides, such as, for example, isothiazolinones, 2-bromo-2-nitro-1,3-propanediol, as described above under “detergents”.
bleaching systems, consisting of bleaches, such as, for example, perborate, percarbonate etc., plus bleach activators, such as, for example, tetraacetylethylenediamine, plus bleach stabilizers, as described above under “detergents”.
Solubilizers, such as, for example cumenesulfonates, toluenesulfonates, short-chain fatty acids, phosphoric alkyl/aryl esters
solvents, such as, for example, short-chain alkyl oligoglycols, such as butyl glycol, butyl diglycol propylene glycol monomethyl ether, alcohols, such as ethanol, isopropanol, aromatic solvents, such as toluene, xylene, N-alkylpyrrolidones, alkylene carbonates.

The constituents of cleaners for hard surfaces are known in principle to the person skilled in the art. The above list represents merely an exemplary section of the constituents.

The cleaners for hard surfaces are usually, but not exclusively, aqueous and are in the form of microemulsions, emulsions or solutions.

Where they are present in solid, pulverulent form, extenders, such as, for example, sodium sulfate, magnesium sulfate, etc. may additionally be used.

In the case of cleaners in the form of tablets, tableting auxiliaries, such as, for example, polyethylene glycols with molar masses >1000 g/mol, polymer dispersions etc., and tablet disintegrants, such as, for example, cellulose derivatives, crosslinked polyvinylpyrrolidone, crosslinked polyacrylates or combinations of acids, e.g. citric acid plus sodium bicarbonate, to name but a few, are additionally required.

Products from the bodycare sector are, for example, shampoos, shower and bath gels, shower and bath lotions, lipsticks and cosmetic formulations with care and/or conditioning properties, such as styling products. Examples are hair foams, hair gels, hair sprays or after-treatment compositions, such as hair tonics, lotions, treatment rinses, treatment packs, split-end fluids, hair repair compositions, “hot oil treatments”, shampoos, liquid soaps, care creams, hair-setting compositions, hair colorants and permanent waving compositions. When used in bodycare products, the substances according to the formulae I and II have the advantage that the physiological irritantancy of the surfactant mixtures is moderated and the mucous membranes are protected.

The invention is now illustrated in the examples below.

### EXAMPLE 1

### 3,5-Bis(1-ethylpentyl)-7a-hydroxymethyldihydrooxazolo[3,4-c]oxazole

0.55 mol of ethylhexanal, 0.25 mol of tris(hydroxymethyl)aminomethane and 0.25 g of Amberlyst 15 are mixed at room temperature. The reaction mixture is heated to 100° C. at 500 mbar. The water which forms distills off over the course of 3 hours. The mixture is then left to cool to room temperature and filtered. The reaction product can be used without further purification.

### EXAMPLE 2

### 3,5-Bis(1-ethylpentyl)-7a-hydroxymethyldihydrooxazolo[3,4-c]oxazole

The reaction was carried out in a distillation apparatus. 256 g (2.0 mol) of 2-ethylhexanal were combined with 121 g (1.0 mol) of tris-hydroxymethylaminomethane at room temperature. The mixture was then heated to 100° C. at a pressure of 500 mbar. After 1 l at 500 mbar/100° C., the pressure is reduced to 250 mbar until distillate no longer passed over. The distillate collected was 2-phase. The phase containing ethylhexanal was returned to the reaction mixture and stirred again for 1 h at 500 bar and 100° C. The product can be used without further purification.

### EXAMPLE 3

### 2-(1-Ethylpentyl)4,4-di(hydroxymethyl)oxazolidine

1.5 mol of 2-ethylhexanal, 1.5 mol of tris(hydroxymethyl)aminomethane and 1.5 g of Amberlyst 15 were mixed at room temperature with 300 ml of toluene. The mixture is boiled for three hours with a water separator until no new water is collected. The mixture is then allowed to cool to room temperature and filtered, and the solvent is evaporated under reduced pressure. The product can be used without further purification.

### EXAMPLE 4

### 7a-Hydroxymethyl-3,5-di(dodecyl)dihydrooxazolo[3,4-c]oxazole/7a-hydroxymethyl-3,5-di(tetradecyl)dihydrooxazolo[3,4-c]oxazole

0.85 mol of C13/C15-aldehyde mixture, 0.39 mol of tris(hydroxymethyl)aminomethane and 0.4 g of Amberlyst 15 are mixed at room temperature. The reaction mixture is heated to 100° C. at 500 mbar. The water which forms is distilled over the course of 3 hours. The mixture is then left to cool to room temperature and filtered. The reaction product can be used without further purification.

### EXAMPLE 5

### 4,4-Di(hydroxymethyl)-2-dodecyloxazolidine/4,4-di(hydroxymethyl)-2-tetradecyloxazolidine

1.5 mol of C13/C15-aldehyde mixture, 1.5 mol of tris(hydroxymethyl)aminomethane and 1.5 g of Amberlyst® 15 were mixed with 300 ml of toluene at room temperature. The mixture is boiled for three hours using a water separator until no more new water is collected. The mixture is then left to cool to room temperature and filtered, and the solvent is evaporated under reduced pressure. The product can be used without further purification.

### EXAMPLE 6

### 2-(1-Ethylpentyl)-[1,3]-dioxolan-4-one

0.50 mol (64.1 g) of 2-ethylhexanal and 0.3 g of Amberlyst® 15 were added to 150 ml of chloroform at room temperature. The water separator was filled with 50 ml of chloroform and the reaction mixture was refluxed (93° C.). 0.50 mol (50.0 g) of lactic acid (90% aqueous solution) was slowly added dropwise and stirred under reflux until 19 ml of H₂O had been separated off. Finally, the Amberlyst® 15 was filtered off and the product was purified by distillation.

### EXAMPLE 7

### Hand Dishwashing Detergent

A model formulation comprising 30% by weight of Lutensit® ALBN50 (BASF AG, alkylbenzenesulfate, 50%), 10% by weight of Lutensol® AO7 (BASF AG, C13/15, alcohol ethoxylate, 7 ethylene oxide, 100%), 3% by weight of 3,5-bis-(1-ethylpentyl)-7a-hydroxymethyldihydrooxazolo[3,4-c]oxazole is admixed with various amounts of Lutensol® A3N (BASF AG, C12,14-alcohol ethoxylate, 3EO, 100% BASF AG). The resulting mixtures are analyzed using an Uhbelohde viscometer, spindle 3, shear rate 3 s⁻¹. In parallel experiments, a corresponding surfactant mixture in which the reaction product was replaced by Mazox® LDA (laurylamine oxide, 100%, origin BASF Corporation) and by water were investigated. The results are summarized in the table. The viscosity increase is most marked for the product according to the invention.

| 0 | 1 | 2 | 4 | 6 | 8 | % Lutensol ® A3N |
|---|---|---|---|---|---|---|
| 892 | 2900 | 6760 | 30000 | 172000 | | 3,5-bis-(1-ethylpentyl)-7a-hydroxy- |
| | | | | | | methyldihydrooxazolo[3,4-c]oxazole |
| 1210 | 905 | 970 | 1820 | 2890 | 7010 | Water |
| 2040 | 2500 | 2910 | 5760 | 12700 | 19200 | Mazox LDA Oxide w.s. |

### EXAMPLE 8

### Hand Dishwashing Detergent

Foam stabilization with 3,5-bis(1-ethylpentyl)-7a-hydroxymethyldihydrooxazolo[3,4-c]oxazole.

A model formulation comprising 30% by weight of Lutensit® ALBN50 (alkylbenzenesulfate, 50%), 10% by weight of Lutensol® AO7 (C13/15, alcohol ethoxylate, 7 ethylene oxide, 100%), 3% by weight of 3,5-bis(1-ethylpentyl)-7a-hydroxymethyldihydrooxazolo[3,4-c]oxazole and 3% by weight of Lutensol A3N (C12,14-alcohol ethoxylate, 3EO, 100%) is diluted to 2% by weight of surfactant. In a beaker (5 l in volume, filled to 2 l), this surfactant solution is added to the foam by stirring. When a stable state has been established, fresh olive oil is added dropwise until the foam has disappeared. The amount of oil necessary for this is a measure of the stability of the foam. In parallel experiments, a corresponding surfactant mixture in which the reaction product was replaced by Mazox® LDA (laurylamine oxide, 100%) and by water were investigated. The results are summarized in the table.

| | Consumption of olive |
|---|---|
| Additive | oil |
| 3,5-Bis(1-ethylpentyl)-7a-hydroxymethyl- | 37 ml |
| dihydrooxazolo[3,4-c]oxazole + 3 EO | |
| Mazox ® LDA | 28 ml |
| Water | 27 ml |

1. A cyclic aldehyde derivative of the formulae 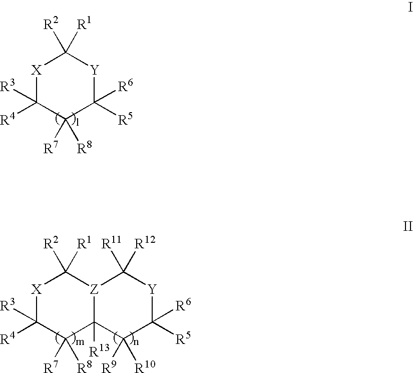
in which the symbols X, Y, Z and R¹ to R¹³ have the following meanings:
R¹ is hydrogen or a linear or branched, substituted or unsubstituted C₃-C₂₉-alkyl group or a linear or branched, substituted or unsubstituted C₃-C₂₉-alkenyl group, where one or more carbon atoms in the alkyl or alkenyl chain may be replaced by —O—, —NR¹⁴, —C(O)NR¹⁵— or —S— and —O—O— and —S—S— are excluded;
R² is hydrogen or —CH₃;
R³, R⁴, R⁵ and R⁶ are, independently of one another, chosen from the group of substituents consisting of: H; —CN; —C(O)OH; —C(O)OR¹⁸; —C(O)NR¹⁹R²⁰; C₆H₅, in which one or more hydrogens may be replaced by substituents; 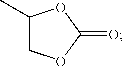
and C₁-C₅-alkyl groups which, at any desired point in the chain, may have 1 to 4 substituents from the group —OH; —SH; —CN; NR¹⁶R¹⁷; —OR²²; or 1 to 2 substituents from the group consisting of —C(O)OH; —C(O)OR¹⁸; —C(O)NR¹⁹R²⁰; —OSO₃⁻; —SO₃⁻; —OPO₃²⁻; OPO(OR²¹)₂; C₆H₅, in which one or more hydrogens may be replaced by substituents; and 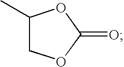
or
one of the substituent pairs R³, R⁴ and R⁵, R⁶ is ═O;
R⁷, R⁸, R⁹ and R¹⁰ are, independently of one another, chosen from the group of substituents consisting of: H; —CN; —NR¹⁶R¹⁷; —C(O)OH; —C(O)OR¹⁸; —C(O)NR¹⁹R²⁰; —OSO₃⁻; —OPO₃²⁻; OPO(OR²¹)₂; C₆H₅, in which one or more hydrogens may be replaced by substituents; 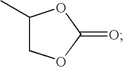
and C₁-C₅-alkyl groups which, at any desired point on the chain, may have 1 to 4 substituents from the group —OH; —SH; —CN; NR¹⁶R¹⁷; —OR²²; or 1 to 2 substituents from the group consisting of —C(O)OH; —C(O)OR¹⁸; —C(O)NR¹⁹R²⁰; —OSO₃⁻; —SO₃³¹ ; —OPO₃²⁻; OPO(OR²¹)₂; C₆H₅, in which one or more hydrogens may be replaced by substituents; and 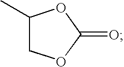
R¹¹ has, independently, the same meaning as R¹;
R¹² has, independently, the same meaning as R²;
R¹³ has, independently, the same meaning as R³, R⁴, R⁵or R⁶;
R¹⁴ is a linear or branched C₁-C₄-alkyl group;
R¹⁵ is hydrogen or a linear or branched C₁-C₄-alkyl group;
R¹⁶, R¹⁷ are, independently of one another, hydrogen or a linear or branched C₁-C₄-alkyl group;
R¹⁸ is chosen from the group consisting of C₁-C₆-alkyl groups and ethyleneoxy groups —(CH₂—CH₂O—)ₚ;
R¹⁹, R²⁰ have, independently, the same meaning as R¹⁶, R¹⁷;
R²¹ is a C₁-C₄-alkyl group or —C₆H₅;
R²² is chosen from the group consisting of C₁-C₁₀-alkyl groups, acyl groups —C(O)R²³ and the group consisting of ethyleneoxy groups —(CH₂—CH₂O—)_{q}, propyleneoxy groups —(CH(CH₃)—CH₂O—)ᵣ, butyleneoxy groups —(C₄H₉O—)ₛ, and alkyleneoxy groups containing at least two of the abovementioned groups in the form of block or random copolymers and containing a total of at most 15 alkyleneoxy units;
R²³ is a C₁-C₁₈-alkyl group;
X and Y in formula I and II are, independently of one another, O, S, or NR²⁴, Z in formula II is N;
R²⁴ is hydrogen or a C₁-C₄-alkyl group;
l, m and n are, independently of one another, 0 or 1;
p is an integer from 1 to 15;
q is an integer from 1 to 15;
r is an integer from 1 to 15;
s is an integer from 1 to 15;
and where the aliphatic moiety of the compounds of the formula I or II which does not originate from the feed aldehyde, in cases where X, Y are ═O and R³, R⁴ or R⁵, R⁶ are ═O, must have at least 2 carbon atoms, and in all other cases must have at least 3 carbon atoms.

2. A compound as claimed in claim 1 in which one, two or more or all of the symbols X, Y and Z, one, two or more or all of the substituents R¹ to R¹³, and one or more of the symbols l, m and n have the following meanings:
R¹ is a linear or branched C₅-C₁₇-alkyl group or a linear or branched C₃-C₁₇-alkenyl group, where one or more carbon atoms in the alkyl chain may be replaced by O or NR¹⁴ and —O—O— is excluded;
R² is —H;
R³, R⁴, R⁵ and R⁶ are, independently of one another, chosen from the group consisting of: —H; —C(O)OH; —C(O)OR¹⁸; 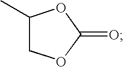
and C₁-C₅-alkyl groups which, at any desired position on the chain, may have 1 or 2 substituents from the group —OH; —CN; NR¹⁶R¹⁷; —OR²²; or 1 substituent from the group consisting of —C(O)OH; —C₆H₅, in which one or more hydrogens may be replaced by substituents; and 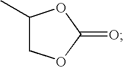
or
one of the substituent pairs R³, R⁴ and R⁵, R⁶ is ═O;
R⁷, R⁸, R⁹ and R¹⁰ are, independently of one another, chosen from the group consisting of: —H; —NR¹⁶R¹⁷; —C(O)OH; —C(O)OR¹⁸; 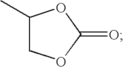
and C₁-C₅-alkyl groups which, at any desired position on the chain, may have 1 or 2 substituents from the group —OH; —CN; NR¹⁶R¹⁷; —OR²²; or 1 substituent from the group consisting of —C(O)OH; —C₆H₅, in which one or more hydrogens may be replaced by substituents; and 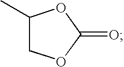
R¹¹ has, independently, the same meaning as R¹;
R¹² has, independently, the same meaning as R²;
R¹³ has, independently, the same meaning as R³, R⁴, R⁵ or R⁶;
R¹⁴ is a linear or branched C₁-C₄-alkyl group;
R¹⁶, R¹⁷ are, independently of one another, hydrogen or a linear or branched C₁-C₄-alkyl group;
R¹⁸ is chosen from the group consisting of C₁-C₆-alkyl groups and ethyleneoxy groups —(CH₂—CH₂O—)ₚ;
R²² is chosen from the group consisting of C₁-C₄-alkyl groups, acyl groups —C(O)R²³ and the group consisting of ethyleneoxy groups —(CH₂—CH₂O—)_{q}, propyleneoxy groups —(CH(CH₃)—CH₂O—)ᵣ and butyleneoxy groups —(C₄H₉O—)ₛ, and mixed alkyleneoxy groups;
R²³ is a C₁-C₁₈-alkyl group;
X and Y in the formula I and II are, independently of one another, O or NR²⁴, Z in formula II is N;
R²⁴ is hydrogen or a C₁-C₄-alkyl group;
l, m and n are, independently of one another, 0 or 1;
p is an integer from 1 to 15;
q is an integer from 1 to 10;
r is an integer from 1 to 10;
s is an integer from 1 to 10;
and where the aliphatic moiety of the compounds of the formula I or II which does not originate from the feed aldehyde, in cases where X, Y are ═O and R³, R⁴ or R⁵, R⁶ are ═O, must have at least 2 carbon atoms, and in all other cases must have at least 3 carbon atoms.

3. A compound as claimed in claim 1 or 2, in which one, two or more or all of the symbols X, Y, Z and l, m, and n and also one, two or more or all of the substituents R¹ to R¹³ have the following meanings:
R¹ is a linear or branched C₅-C₁₇-alkyl group or a linear or branched C₅-C₁₇-alkenyl group;
R² is —H;
R³, R⁴, R⁵ and R⁶ are, independently of one another, chosen from the group consisting of: —H; —C(O)OH; and C₁-C₃-alkyl groups which can have, at any desired point on the chain, 1 or 2 substituents from the group —OH; —NR¹⁶R¹⁷; —OR²²; or 1 substituent from the group consisting of —C(O)OH; and 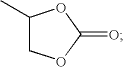
or
one of the substituent pairs R³, R⁴ and R⁵, R⁶ is ═O;
R⁷, R⁸, R⁹ and R¹⁰ are, independently of one another, chosen from the group consisting of: —H; —NR¹⁶R¹⁷; —C(O)OH; and C₁-C₃-alkyl groups which, at any desired point on the chain, can have 1 or 2 substituents from the group —OH; NR¹⁶R¹⁷; or 1 substituent from the group consisting of —C(O)OH; and 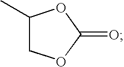
R¹¹ has, independently, the same meaning as R¹;
R¹² has, independently, the same meaning as R²;
R¹³ has, independently, the same meaning as R³, R⁴, R⁵ or R⁶;
R¹⁶, R¹⁷ are, independently of one another, hydrogen or a linear or branched C₁-C₄-alkyl group;
R²² is chosen from the group consisting of C₁-C₄-alkyl groups, acyl groups —C(O)R²³ and the group consisting of ethyleneoxy groups —(CH₂—CH₂O—)_{q}, propyleneoxy groups —(CH(CH₃)—CH₂O—)ᵣ and butyleneoxy groups —(CH₂CH(C₂H₅—O—)s-, and mixed alkyleneoxy groups;
R²³ is a C₁-C₁₈-alkyl group;
X and Y in formula I and II are, independently of one another, O or NR²⁴, Z in formula II is N;
R²⁴ is hydrogen or a C₁-C₄-alkyl group;
l, m and n are, independently of one another, 0 or 1;
q is an integer from 1 to 10;
r is an integer from 1 to 10;
s is an integer from 1 to 10;
and where the aliphatic moiety of the compounds of the formula I or II which does not originate from the feed aldehyde in cases where X, Y are ═O and R³, R⁴ or R⁵, R⁶ are ═O must have at least 2 carbon atoms, and in all other cases must have at least 3 carbon atoms.

4. A compound as claimed in any of claims 1 to 3, in which one, two or more or all of the symbols X, Y, Z and l, m, and n and also one, two or more or all of the substituents R¹ to R¹³ have the following meanings:
R¹ is a linear or branched C₃-C₂₁-alkyl group or a linear or branched C₃-C₂₁-alkenyl group;
R² is —H;
R³, R⁴, R⁵ and R⁶ are, independently of one another, chosen from the group consisting of: —H; —C(O)OH; and C₁-C₃-alkyl groups which may have, at any desired point on the chain, 1 or 2 substituents from the group —OH; —NR¹⁶R¹⁷; —OR²²; or 1 substituent of the type —C(O)OH; or
one of the substituent pairs R³, R⁴ and R⁵, R⁶ is ═O;
R⁷, R⁸, R⁹ and R¹⁰ are, independently of one another, chosen from the group consisting of: —H; —NR¹⁶R¹⁷; —C(O)OH; and C₁-C₃-alkyl groups which may have, at any desired point on the chain, 1 or 2 substituents on the group —OH; NR¹⁶R¹⁷; or 1 substituent of the type —C(O)OH;
R¹¹ has, independently, the same meaning as R¹;
R¹² has, independently, the same meaning as R²;
R¹³ has, independently, the same meaning as R³, R⁴, R⁵ or R⁶;
R¹⁶, R¹⁷ are, independently of one another, hydrogen or a linear or branched C₁-C₄-alkyl group;
R²² is chosen from the group consisting of C₁-C₄-alkyl groups and ethyleneoxy groups —(CH₂—CH₂O—)_{q};
X and Y in formula I and II are, independently of one another, O, S, or NR²⁴, Z in formula II is N;
R²⁴ is hydrogen or a C₁-C₄-alkyl group;
l, m and n are, independently of one another, 0 or 1;
q is an integer from 3 to 8;
and where the aliphatic moiety of the compounds of the formula I or II which does not originate from the feed aldehyde in cases where X, Y are ═O and R³, R⁴ or R⁵, R⁶ are ═O must have at least 2 carbon atoms, and in all other cases must have have at least 3 carbon atoms.

5. As compound as claimed in claim 1:
2-(1-ethylpentyl)-[1,3]-dioxolan-4-one (1);
3,5-bis(1-ethylpentyl)-7a-hydroxymethyldihydrooxazolo[3,4-c]oxazole (2);
7a-hydroxymethyl-3,5-di(nonyl)dihydrooxazolo[3,4-c]oxazole (3);
2-(1-ethylpentyl)4,4-di(hydroxymethyl)oxazolidine (4);
4,4-di(hydroxymethyl)-2-nonyloxazolidine (5);
2-(1-propylhexyl)4,4-di(hydroxymethyl)oxazolidine (6);
7a-hydroxymethyl-3,5-di(dodecyl)dihydrooxazolo[3,4-c]oxazole (7);
7a-hydroxymethyl-3,5-di(tetradecyl)dihydrooxazolo[3,4-c]oxazole (8);
7a-hydroxymethyl-3,5-di(undecyl)dihydrooxazolo[3,4-c]oxazole (9);
7a-hydroxymethyl-3,5-di(tridecyl)dihydrooxazolo[3,4-c]oxazole (10);
4,4-di(hydroxymethyl)-2-dodecyloxazolidine (11);
4,4-di(hydroxymethyl)-2-tetradecyloxazolidine (12);
4,4-di(hydroxymethyl)-2-undecyloxazolidine (13);
4,4-di(hydroxymethyl)-2-tridecyloxazolidine (14);
2-(1-propylhexyl)-[1,3]-dioxolan-4-one (15)
2-(1-propylhexenyl)-[1,3]-dioxolan-4-one (16)
7a-hydroxymethyl-3,5-di(1-propylhexenyl)dihydrooxazolo[3,4-c]oxazole (17)
4,4-di(hydroxymethyl)-2-(1-propylhexenyl)oxazolidine (18)
mixtures of (7) and (8);
mixtures of (11) and (12)
mixtures of (9) and (10);
mixtures of (13) and (14);
adducts of one of the substances (1) to (18) having 3 to 10 ethylene oxide units and mixtures thereof.

6. A compound as claimed in any of claims 1 to 5, wherein the substituent R¹ has an average degree of branching from 0 to 2.5, preferably 0.2 to 1.6.

7. A process for the preparation of a compound as claimed in any of claims 1 to 6, which comprises, in a condensation reaction known per se, reacting the respective aldehyde or an aldehyde mixture and the polyfunctional compound to be reacted therewith and comprising at least two identical or different functional groups chosen from hydroxyl, diol, carboxyl and primary and secondary amino functions or a mixture of polyfunctional compounds, together, optionally in the presence of a suitable acid.

8. A process as claimed in claim 7, wherein Lewis or Brönsted acids known per se are used in liquid or solid form, preferably sulfuric acid, p-toluenesulfonic acid, p-toluenesulfonic acid pyridinium salt or acidic ion exchangers.

9. A process as claimed in claim 7 or 8, wherein the aldehyde used is a linear or branched aliphatic C₄-C₃₀-aldehyde, preferably a C₆-C₁₈-aldehyde, having an average degree of branching of from 0 to 2.5, preferably 0.2 to 1.6.

10. A process as claimed in any of claims 7 to 9, wherein the polyfunctional compound is a compound chosen from the following groups:
linear and branched aliphatic C₃-C₆-polyols with at least two hydroxyl functions, preferably 2 to 5 hydroxyl functions, in particular 2 to 4 hydroxyl functions, in which, in addition to the hydroxyl functions, further functional groups may be present which are chosen from the group —SH; —CN; NR¹⁶R¹⁷; —C(O)OH; —C(O)OR¹⁸; —C(O)NR¹⁹R²⁰; —OSO₃⁻; —SO₃⁻; OPO₃²⁻; OPO(OR²¹)₂; —OR²²; C₆H₅, in which one or more hydrogens may be replaced by substituents; and 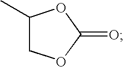
linear and branched aliphatic C₃-C₆-alkanolamines with at least one primary or secondary amino function and a hydroxyl function, in which a maximum of 4 further hydroxyl or amino functions may be present, preferably exactly one amino function, and further substituents may be present from the group: —SH; —CN; —C(O)OH; —C(O)OR¹⁸; —C(O)NR¹⁹R²⁰; —OSO₃⁻; —SO₃⁻; —OPO₃²⁻; OPO(OR²¹)₂; —OR²²; —C₆H₅, in which one or more hydrogens may be replaced by substituents; and 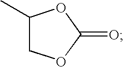
linear and branched aliphatic C₃-C₆-thiols with one thiol function and one hydroxyl function, in which four further hydroxyl or thiol functions may be present, preferably the thiol has exactly one SH function and further substituents may be present from the group: —OH; —SH; —CN; NR¹⁶R¹⁷; —C(O)OH; —C(O)OR¹⁸; —C(O)NR¹⁹⁵R²⁰; —OSO₃⁻; —SO₃⁻; —OPO₃²⁻; OPO(OR²¹)₂; —OR²²; —C₆H₅, in which one or more hydrogens may be replaced by substituents; and 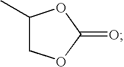
linear and branched aliphatic C₃-C₆-hydroxycarboxylic acids with one hydroxyl function and one carboxyl function in which four further hydroxyl or carboxyl functions may be present, preferably the molecule has exactly one carboxyl function and further substituents may be present from the group: —SH; —CN; NR¹⁶R¹⁷; —C(O)OR¹⁸; —C(O)NR¹⁹R²⁰; —OSO₃⁻; —SO₃⁻; —OPO₃²⁻; OPO(OR²¹)₂; —OR²²; —C₆H₅, in which one or more hydrogens may be replaced by substituents; and 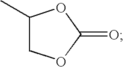
linear and branched aliphatic C₃-C₆-diamines with 2 to 6 primary or secondary amino functions, preferably 2 to 4 primary or secondary amino functions, and further substituents may be present from the group: —OH; —SH; —CN; —C(O)OH; —C(O)OR¹⁸; —C(O)NR¹⁹R²⁰; —OSO₃⁻; —SO₃⁻; —OPO₃²⁻; OPO(OR²¹)₂; —OR²²; —C₆H₅, in which one or more hydrogens may be replaced by substituents; and 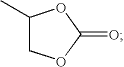
linear and branched aliphatic C₃-C₆-aminothiols with at least one primary or secondary amino function and one thiol function, preferably the aminothiol used has exactly one primary or secondary amino function and exactly one thiol function, and further substituents may be present from the group: —OH; —CN; —C(O)OH; —C(O)OR¹⁸; —C(O)NR¹⁹R²⁰; —OSO₃⁻; —SO₃⁻; —OPO₃²⁻; OPO(OR²¹)₂; —OR²²; —C₆H₅, in which one or more hydrogens may be replaced by substituents; and 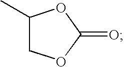
linear and branched aliphatic C₃-C₆-amino acids with at least one primary or secondary amino function and a carboxyl function, preferably exactly one primary or secondary amino function;
linear and branched aliphatic C₃-C₆-dithiols with at least two thiol functions, preferably exactly two thiol functions, and further substituents may be present from the group: —OH; —CN; NR¹⁶R¹⁷; —C(O)OH; —C(O)OR¹⁸; —C(O)NR¹⁹R²⁰; —OSO₃⁻; —SO₃—; —OPO₃²⁻; OPO(OR²¹)₂; —OR²²; —C₆H₅, in which one or more hydrogens may be replaced by substituents; and

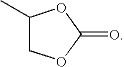

11. A process as claimed in any of claims 7 to 10, wherein the polyol used is glycerol, tartaric acid, diethyl tartrate, trimethylolpropane, fructose, cyclohexanediol, sucrose, tetrahydroxybutane, the alkanolamine used is trimethylolmethylamine, diethanolamine, propanolamine, dipropanolamine, amino sugars, the thiol used is mercaptoethanol, mercaptolactic acid, mercaptoglycolic acid, thiosalicylic acid, mercaptosuccinic acid, 3-mercapto-1,2-propanediol, cysteine, N-acetylcysteine, 3-mercaptopropionic acid, penicillamine, the hydroxycarboxylic acid used is lactic acid, citric acid, glycolic acid, tartaric acid, glyceric acid, maleic acid, salicylic acid, the diamine used is propylenediamine, diethylenetriamine, triethylenetetramine, N-aminopropylethylenediamine (N₃-amine), N,N′-bis(aminopropyl)ethylenediamine (N₄ amine), hydroxyethylethylenediamine, the aminothiol used is cysteine, tyrosine, mercaptopropylamine, the amino acid used is aminodiacetic acid HN(CH₂CO₂H)₂, ethylenediaminetriacetic acid, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or N-phosphonomethylglycine.

12. The use of a substance as claimed in any of claims 1 to 6 or a mixture thereof as cosurfactant.

13. A household detergent, household cleaner, body-cleansing composition or bodycare composition comprising at least one compound as claimed in any of claims 1 to 6.

14. A detergent as claimed in claim 13 in solid, liquid, gel or paste form, preferably in the form of a powder, compact, granulate, tablet or gel.

15. A detergent as claimed in claim 13 or 14 comprising 0.1 to 40% by weight, in particular 0.5 to 30% by weight, very particularly 1 to 20% by weight, based on the total amount of the formulation, of at least one compound as claimed in any of claims 1 to 6.

16. A household cleaner as claimed in claim 13 in liquid, gel, or solid form, preferably in the form of a liquid, gel, powder or compact.

17. A household cleaner as claimed in claim 16 in the form of a hand dishwashing detergent, machine dishwashing detergent, metal degreaser, glass cleaner, floor cleaner, all-purpose cleaner, high-pressure cleaner, alkaline cleaner, acidic cleaner, spray degreaser, dairy cleaner, upholstery cleaner, plastics cleaner and bath cleaner.

18. A household cleaner as claimed in claim 16 or 17 comprising 0.01 to 40% by weight, preferably 0.1 to 25% by weight, based on the overall formulation, of at least one substance as claimed in any of claims 1 to 5.

19. A body-cleansing composition or bodycare composition in the form of a shampoo, shower gel or bath gel, shower lotion or bath lotion, a lipstick, a cosmetic formulation with care and/or conditioning properties or a styling product, in particular a liquid soap, a care cream, a hair foam, hair gel, hairspray or after-treatment composition, a hair tonic, a lotion, treatment rinse, treatment pack, split-end fluid, hair repair composition, “hot oil treatments”, hair-setting composition, hair colorant or permanent waving composition.
